(19) United States
(12) Patent Application Publication
(10) Pub. No.: US 2008/0292592 A1
(43) Pub. Date: Nov. 27, 2008

(54) Oncolytic Adenovirus Armed with Therapeutic Genes

(76) Inventors: Sunil Chada, Missouri City, TX (US); William Wold, Chesterfield, MO (US); Lou Zumstein, Del Mar, CA (US)

Correspondence Address: FULBRIGHT & JAWORSKI L.L.P., 600 CONGRESS AVE., SUITE 2400, AUSTIN, TX, 78701, US

(21) Appl. No.: 11/568,499

(22) PCT Filed: May 2, 2005
(86) PCT No.: PCT/US05/15157

§ 371 Date: Aug. 15, 2007

Related U.S. Application Data
(60) Provisional application No. 60/566,674, filed on Apr. 30, 2004.

(51) Int. Cl. A61K 35/76; C12N 7/00; A61P 31/00

(52) U.S. Cl. 424/93.2; 435/235.1

(57) ABSTRACT

The present invention involves compositions and methods for treating preventing cancer using compositions including replication competent adenovirus. The application competent adenovirus may or may not encode a therapeutic polynucleotide.

This application claims priority to U.S. Provisional application Ser. No. 60/566,674, filed Apr. 30, 2004, which is incorporated herein by reference in its entirety

### FIELD OF BACKGROUND OF THE INVENTION

I. Field of the Invention

The invention generally relates to the oncology and oncolytic adenoviruses. More particularly, it concerns compositions and methods of treating cancer in a patient using oncolytic adenoviruses armed with nucleic acids encoding therapeutic polypeptides.

II. Background

Vectors based on adenovirus (Ad) serotype 5 (Ad5) are well suited for cancer gene therapy. Ad5 primarily causes mild or asymptomatic infections in young children that lead to long-lasting immunity. Fatalities in adults are rare and are mostly limited to severely immunocompromised individuals. Ad5 biology is well understood at the molecular level allowing manipulation of the genome, including insertion of foreign sequences. Ads are not oncogenic in humans and their genomic DNA is not integrated into the host genome. Stable, high-titer stocks are easily produced in cell culture, and Ad5 replicates in most human cancer cell types.

Ad5-based vectors are classified as either replication deficient (RD) or replication competent (RC) in normal cells depending on the absence or presence, respectively, of transcription units encoding functional proteins such as the E1A and E1B (Crystal, 1999; Ring, 2002), E2, or E4 proteins. Most RD vectors also delete the E3 transcription unit, which encodes proteins primarily involved in protecting infected cells from attack by the host immune system (Lichtenstein et al., 2004). RD vectors are normally “armed” by inserting a therapeutic gene into the deleted E1, or E3 or other adenoviral region(s).

Although RD Ad vectors can be efficient at killing infected cells, and in some cases neighboring cells, they cannot spread through a tumor, and probably infect only a fraction of the cells within a tumor. In contrast, RC vectors, which kill infected cells because Ad infections are lytic, should, in theory, be capable of multiple rounds of replication leading to spread of the vector through the tumor and consequently enhanced tumor cell lysis. Preclinical studies with RC vectors have shown great promise, but thus far, they have demonstrated limited success in the clinic. The first and most extensively studied RC vector used in the clinic is ONYX-015 (first known as d11520) (Barker and Berk, 1987). Because this vector has a deletion of the E1B55K gene, its replication is in theory limited to cancer cells. However, this deletion also attenuates vector replication (Barker and Berk, 1987), probably limiting the ability of the vector to spread through the tumor and likely explaining the vector's limited clinical success.

Phase I and II clinical trials have now been performed with other RC Ad vectors. The vector Ad5-CD/TKrep, like ONYX-015, contains a deletion of the E1B55K gene, but this vector also expresses a cytosine deaminase (CD)/herpes simplex virus thymidine kinase (HSV-TK) fusion gene, which allows for suicide therapy with both 5-fluorocytosine and gancyclovir (Freytag et al., 1998). A Phase I clinical trial showed that Ad5-CD/TKrep is well tolerated (Freytag et al., 2002). The vectors CG7060 (formerly CV706) (Rodriguez et al., 1997) and CG7870 (formerly CV787) (Yu et al., 1999) are both RC vectors in which a prostate-specific promoter, either the prostate-specific antigen (PSA) enhancer in the former case or the rat probasin promoter in the latter case, replaces the E1A promoter. In CG7870, the E1B promoter is replaced with the PSA enhancer (Yu et al., 1999). A Phase I clinical trial with CG7060 showed it is well tolerated following intraprostatic injection (DeWeese et al., 2001). In a Phase I/II clinical trial with CV7870 the vector resulted in only one grade 3 adverse event at the highest dose (1×10¹³ particles) and no grade 3 or 4 toxicities at lower doses (DeWeese et al., 2003). The efficacy of these vectors awaits the results of additional clinical trials.

The lack of effectiveness with ONYX-015 and the preliminary nature of the other vectors indicates that improvements in vector design and potency can be developed for successful application of RC Ad vectors as a treatment of cancer. A major objective in designing an oncolytic vector should be its rapid and efficient spread through the tumor so that as many cells as possible can be killed.

### SUMMARY OF THE INVENTION

The present invention is based on the observation that an RC Ad vector that expresses a therapeutic polypeptides, such as a tumor suppressor, can be produced and employed as an effective cancer therapeutic agent. Tumor suppressor genes that can be employed in the context of methods and compositions of the invention include, but are not limited to, p53, FHIT, MDA7, FUS1 and PTEN. Embodiments of the invention include an oncolytic adenovirus comprising a nucleic acid encoding an adenovirus death protein (ADP) that is overexpressed in an infected cell, and a nucleic acid encoding a tumor suppressor, wherein all or part of the adenovirus E3 region is deleted. “Overexpression” refers to the cellular production of a greater number of molecules of ADP per genome as compared to wild-type Ad5 at any time in the adenovirus replication cycle beginning at the time of first synthesis of the ADP protein in infected cells, and continuing through the processes of the assembly of infectious virus particles in the cell, the lysis of the infected cell (including an increase in the permeability of the nuclear envelope and plasma membrane), and the release of adenovirus from the infected cell. Preferably, there will be a greater number of ADP protein molecules made from mRNAs that were derived from the adenovirus major late promoter at the time period immediately following the interaction of adenovirus DNA replication, which generally occurs at about 7 hours post-infection (p.i.), and extending to 24 h, 30 h, or even later times post-infection—such as 36 h, 40 h, etc. Preferably also, there will be a greater number of ADP protein molecules made from mRNAs that were derived from the adenovirus early E3 promoter whose expression begins within 1-3 hours following initial infection and is continuous throughout the replication cycle. The number of molecules of ADP may be estimated by conventional direct means using antisera specific to ADP and methods such as (1) western blot followed by quantitation of the ADP protein bands, (2) metabolic radiolabeling of ADP followed by immunoprecipitation, sodium dodecylsulfate polyacrylamide gel electrophoresis followed by quantitation of the ADP protein bands, (3) indirect immunofluorescence, and (4) ELISA. In certain embodiments, the tumor suppressor is selected from the group consisting of p53, FHIT, MDA7, FUS-1 and PTEN. In specific embodiments, the oncolytic adenovirus expresses an MDA7 tumor suppressor. In certain embodiments, the oncolytic adenovirus is VRX-007 (GZ3) or VRX-011 (GZ3-tert) encoding a tumor suppressor.

Further embodiments of the invention include an adenoviral composition comprising a replication competent adenovirus comprising i) an adenoviral death protein gene that is overexpressed in infected cells, and ii) a first therapeutic nucleic acid, wherein all or part of E3 region is deleted; and a second adenovirus comprising a second therapeutic nucleic acid. In certain embodiments, the first therapeutic nucleic acid encodes a tumor suppressor, such as p53, FHIT, MDA7, FUS-1 or PTEN. In specific embodiments, the tumor suppressor is p53. The second therapeutic nucleic acid may also encode a tumor suppressor, such as MDA-7. The first therapeutic nucleic acid and the second therapeutic nucleic acid may encode the same or different therapeutic nucleic acids. In certain aspects of the invention the second adenovirus encodes a replication defective, a replication competent or a conditionally replicating adenovirus.

In some embodiments, the nucleic acid encoding the tumor suppressor is under the control of a heterologous promoter. The promoter may be a constitutive or an inducible promoter. In certain aspects, the promoter is selected from the group consisting of an adenoviral major late promoter (MLP), CMV IE promoter, dectin-1 promoter, dectin-2 promoter, human CD11c promoter, F4/80 promoter, SM22α MHC class II promoter, SV40 promoter, hTERT promoter, polyoma promoter, and adenovirus 2 promoter. It is further contemplated that the expression of ADP may be under the control of any of these promoters. Furthermore, in some cases, the expression of ADP and the tumor suppressor are controlled by the same promoter. Compositions of the invention may be comprised in an pharmaceutically acceptable composition and may further comprise protamine.

In still further embodiments the invention includes a cell comprising an adenoviral composition of the invention. Cells of the invention comprise an oncolytic adenovirus comprising a nucleic acid encoding an adenovirus death protein (ADP) that is overexpressed in an infected cell, and a nucleic acid encoding a tumor suppressor, wherein all or part of the adenovirus E3 region is deleted. In some aspects, a cell has a first adenoviral nucleic acid encoding a replication competent adenovirus and a second adenoviral nucleic acid encoding a replication deficient adenovirus. The first, second or both adenoviral nucleic acids may encode the same or different tumor suppressor. In certain aspects of the invention, the cell is a pre-cancer or cancer cell. Cells of the invention may be eukaryotic, and more particularly, of mammalian origin, such as human cells.

Embodiments of the invention include methods of treating a patient with a hyperproliferative disorder comprising administering to a patient an effective amount of an oncolytic adenovirus described herein. In certain aspects, an individual need not be diagnosed with a hyperproliferative condition, but may be susceptible to or predisposed to a cancerous or hyperproliferative condition. Thus, in this aspect of the invention one may prophylactically treat an individual with compositions of the invention. The therapeutic nucleic acid encoded by an oncolytic adenovirus of the invention may be under the control of an adenoviral or heterologous promoter. The adenoviral promoter may be the major late promoter (Ad-MLP). In certain aspects the therapeutic nucleic acid encodes a tumor suppressor, such as MDA-7, p53, FUS-1, PTEN, or FHIT. In farther embodiments, other therapeutic nucleic acids known to those skilled in the art of gene therapy may also be utilized. In practice of the inventive methods, an oncolytic adenovirus is typically dispersed in a pharmacologically acceptable formulation. The formulation may be administered by injection, perfusion, inhalation, oral or topical application, as well as by other modes of administration of such medicaments known in the art. Administration may occur more than once, twice, three times, four times or more times. In particular aspects of the invention the composition or formulation is administered at least 1, 2, or 3 times to the patient.

In certain embodiments, the methods may further comprise administering to the patient a second therapy, wherein the second therapy is chemotherapy, immunotherapy, surgery, radiotherapy, biological therapy, cryotherapy, hyperthermia, ultrasound, immunosuppressive agents, or a second gene therapy with a therapeutic polynucleotide. In a preferred embodiment, the second therapy is a second gene therapy. The second gene therapy may comprise administration of an effective amount of a second adenoviral composition, such as a replication defective adenovirus. The second therapy may be administered to the patient before, during, after or concurrently with administration of the composition of the invention. In certain aspects of the invention, chemotherapy comprises an alkylating agent, mitotic inhibitor, antibiotic, or antimetabolite. In preferred embodiments, the chemotherapy may comprise CPT-11, temozolomide, taxanes or a platin compound. In further aspects, radiotherapy may comprise X-ray irradiation, UV-irradiation, γ-irradiation, or microwaves. The methods may include administration of about 10³ to about 10¹⁵ viral particles to the patient. More preferably about 10⁵ to about 10¹² viral particles, and most preferably about 10⁸ to about 10¹² viral particles are administered to the patient. The hyperproliferative disorder may be a precancerous condition, such as cellular hyperplasia, adenoma, metaplasia, or dysplasia. In additional aspects, the hyperproliferative disorder may be cancer, such as a carcinoma, a sarcoma, a metastatic cancer, a lymphatic metastases, a blood cell malignancy, a multiple myeloma, an acute leukemia, a chronic leukemia, a lymphoma, a head and neck cancer, a mouth cancer, a larynx cancer, a thyroid cancer, a respiratory tract cancer, a lung cancer, a small cell carcinoma, a non-small cell cancer, a breast cancer, ductal carcinoma, gastrointestinal cancer, esophageal cancer, stomach cancer, colon cancer, colorectal cancer, pancreatic cancer, liver cancer, genitourinary cancer, urologic cancer, bladder cancer, prostate cancer, ovarian carcinoma, uterine cancer, endometrial cancer, kidney cancer, renal cell carcinoma, brain cancer, neuroblastoma, astrocytic brain tumors, gliomas, metastatic tumor cell invasion in the central nervous system, bone cancers, osteomas, skin cancer, malignant melanoma, squamous cell carcinoma, basal cell carcinoma, hemangiopericytoma or Kaposi's sarcoma. The cancer may be a recurrent cancer, a refractory cancer, or a metastasis.

Embodiments discussed in the context of a methods and/or composition of the invention may be employed with respect to any other method or composition described herein. Thus, an embodiment pertaining to one method or composition may be applied to other methods and compositions of the invention as well.

“A” or “an,” as used herein in the specification, may mean one or more than one. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIG. 1 Schematic of Ad5, KD3, VRX-011 and VRX-007. The horizontal bar indicates the double-stranded Ad5 DNA genome of ˜36 kbp encoding about 34 genes. The arrows indicate transcription units. The “immediate early” E1A proteins induce expression of the “delayed early” proteins encoded by the E1B, E2, E3, and E4 transcription units. Viral DNA begins to replicate at about 7 hours (h) postinfection (p.i.) then “late” proteins derived from the major late transcription unit are synthesized. Alternative splicing and polyadenylation of a large pre-mRNA initiated at the single major late promoter and extending to the right end of the genome from the major late mRNAs. All late mRNAs have a tripartite leader (leaders 1, 2, and 3) at their 5′ termini that facilitates translation. Beginning at 20-24 h p.i., virions begin to assemble in the nucleus, then after 2-3 days the cells begin to lyse and release virions, with lysis complete by about 5-6 days. ADP, predominantly a late protein derived from the major late transcription unit, mediates efficient cell lysis.

FIG. 2 VRX-007 suppresses the growth of A549 human lung tumors in nude mice. Nude mice were injected subcutaneously with 1×10⁷ A549 lung cancer cells into both hind flanks. When tumors reached about 100 μl, the mice were injected intratumorally with vehicle only (Mock) or 3×10⁸ plaque forming units (PFU) of VRX-007 on days 0, 1, and 2. The graph shows the median tumor growth from the injection of virus (Day 0). Significance was p=0.01 by Student's T-test

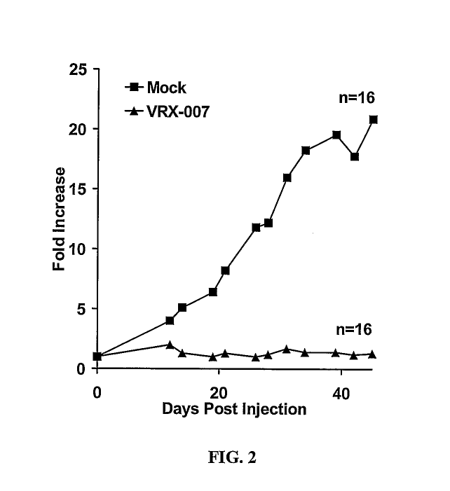

FIGS. 3A-3B Intravenous administration of VRX-007 causes the regression of subcutaneous (FIG. 3A) LNCaP prostate or (FIG. 3B) Hep3B liver tumor xenografts in nude mice. Athymic nude mice were injected subcutaneously with cancer cells into both hind flanks. When tumors reached about 200 μl, the mice were injected intravenously with vehicle (Mock) or VRX-007 on days 0, 1, and 2 with ⅓ of the total dose of vector. The total dose of vector used was (FIG. 3A) 3×10⁸ PFU or (FIG. 3B) 3×10⁷, 9×10⁷, or 3×10⁸ PFU of VRX-007. For the LNCaP experiment Mock n=8 and VRX-007 n=12. For the Hep3B experiment Mock n=9, 3×10⁷ of VRX-007 n=13, 9×10⁷ of VRX-007 n=14, and 3×10⁸ of VRX-007 n=8. Significance was (A) p<0.01 or (13) p=0.01 for all VRX-007 doses groups vs. mock (Student's t-test).

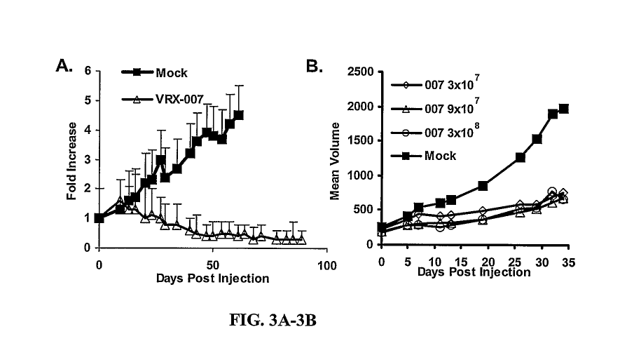

FIG. 4 Ads replicate in LCRT cells. LCRT cells were infected with 50 PFU/cell of Ad5 or VRX-007. After infection, the cells were washed three times with medium. Cells and medium were harvested together at daily intervals from 0 to 8 days post infection. Cells were lysed, and the harvested samples were titered on A549 human lung cells.

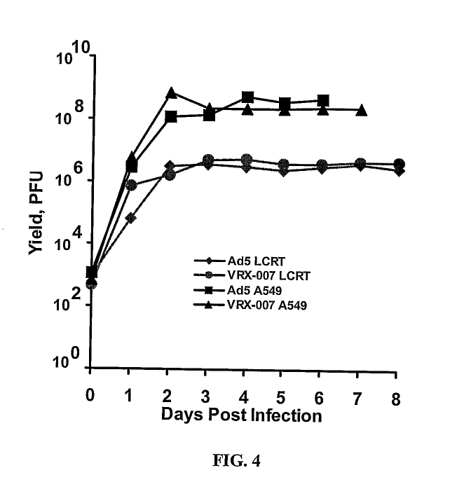

FIG. 5 LCRT cells pre-infected with VRX-007 show reduced tumor growth. LCRT cells in culture dishes were infected with 100 PFU/cell of VRX-007 (“100% infected”). Some infected cells were mixed with uninfected cells (“20% infected”). Control groups mock infected cells (“Mock”), and mock infected cells that had been killed by freezing and thawing (“dead Mock”). Each flank was injected with 4×10⁶ LCRT cells of one of the samples. Tumor volume was measured periodically using a digital caliper and data were analyzed using SPSS© software. The median, interquartile range, and standard deviation of tumor volumes at twelve days post injection are shown.

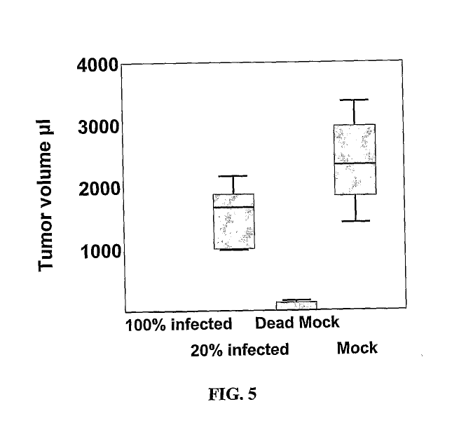

FIG. 6 Intratumoral injection of VRX-007 delays the development of subcutaneous LCRT tumor transplants in cotton rats. Cotton rats bearing LCRT tumors were mock injected or injected with a total dose of 3.4×10¹¹ VP of intact or UV inactivated VRX-007. The tumors were measured on the indicated days after injection of cells. The circles denote outliers (volumes between 1.5 and 3 box lengths from edge of box). The asterisks are extreme outliers (volumes more than 3 box lengths from box edge). The significance was tested by one-way ANOVA and Student's t-Test (VRX-007 vs. Mock or UV inactivated p<0.001).

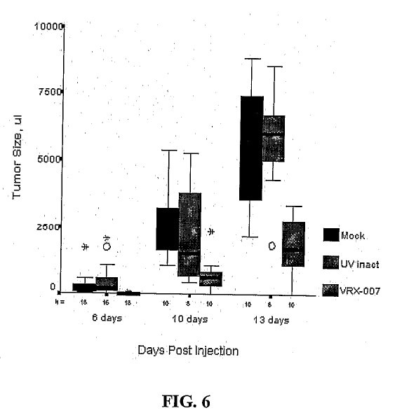

### DETAILED DESCRIPTION OF THE INVENTION

Adenovirus (Ad) vectors are being developed as anti-cancer agents. Most of the Ad vectors developed to date are replication defective (RD) meaning that they must be “armed” with, i.e., encode, a heterologous therapeutic transgene in order to kill infected cells. Since they cannot replicate and infect additional cells, most RD vectors kill only those cells originally infected. More recently, replication competent (RC) Ad vectors, which kill cells due to the lytic nature of the Ad replication cycle, have been developed. Theoretically, RC vectors should be more efficacious than RD vectors because the former should spread through the tumor by means of multiple rounds of infection. To enhance their efficacy, RC vectors can be “armed” with a therapeutic polynucleotide, e.g., a heterologous therapeutic transgene; this introduces another mechanism for killing cancer cells. An alternative approach is to co-infect cells with RC and RD Ad vectors. In a “multi-vector” strategy, not only does the RC Ad vector lyse infected cells but it also serves to replicate the RD vector, thereby amplifying transgene expression and enhancing spread of the RD vector. Furthermore, with a multi-vector approach the potential exists for a multi-modal attack on the tumor by using a single RC Ad vector with multiple RD Ad vectors simultaneously, each with a different mode of action.

### I. Armed Oncolytic Adenovirus and Related Compositions

The VRX-007 vector, also known as GZ3, includes two beneficial features: 1) it retains the wild-type E1A and E1B genes, to achieve maximal vector replication, and 2) it overexpresses the Adenovirus Death Protein (ADP), to enhance vector spread. ADP (formerly E3-11.6K) (Wold et al., 1984, also see U.S. Pat. No. 6,627,190, which is incorporated herein by reference in its entirety) is an integral membrane protein that is localized to the nuclear envelope and endoplasmic reticulum (Scaria et al., 1992), is synthesized mainly late in infection (Tollefson et al, 1992), and is required for efficient virus release and spread (Tollefson et al., 1996a; Tollefson et al., 1996b; Tollefson et al., 2003; Ying and Wold, 2003) of subgroup C Ads. A therapeutic adenovirus may exhibit an upregulated expression of ADP relative to wild-type adenovirus. It has been shown that overexpression of ADP in the context of a RC Ad vector increases vector spread through cultured cells and improves the efficacy of the vector in xenograft tumor models (Toth et al., 2003; Doronin et al., 2000; Doronin et al., 2001; Doronin et al., 2003). Subsequently, another group included ADP in their vector and had similar success in improving vector spread (Ramachandra et al., 2001). Vectors that include the entire E3 region also spread more rapidly than vectors which lack the adp gene, undoubtedly due to the presence of the adp gene (Yu et al., 1999; Suzuki et al, 2002).

Even with improvements in vector design that enhance vector replication and spread, it is unlikely that the vector will kill all cells within a tumor. Physical barriers posed by the architecture of the tumor, down-regulation of CAR (the Ad receptor) in tumor cells, and intrinsic resistance to infection by cells are mechanisms that may prevent elimination of the entire tumor. For these reasons, it would be beneficial to express a therapeutic protein or polynucleotide that acts through a different mechanism from Ad-induced cytolysis. The transgene can either be incorporated into the vector itself creating an armed vector (Hermiston and Kuhn, 2002), or it could be expressed by co-infecting tumor cells with RC and RD vectors, either of which may be armed with the other vector not containing a transgene.

A series of RC Ad vectors have been created in which a therapeutic transgene was inserted into different parts of the E3 transcription unit (U.S. Provisional Application Ser. No. 60/458,493; Hawkins and Hermiston, 2001; Hawkins et al, 2001; each of which is incorporated by reference herein in its entirety). These vectors take advantage of the natural E3 promoter, splice sites, and polyadenylation signals by replacing an endogenous E3 gene(s) with a single transgene. In most cases, the temporal regulation pattern was retained when the endogenous gene was replaced by the transgene. More recently, the vector ONYX-372 was created in which two therapeutic transgenes were simultaneously expressed in the correct temporal pattern when inserted into two different regions of the E3 transcription unit (Bauzon et al., 2003). This vector shows that RC Ad vectors can be armed with more than one therapeutic transgene, both of which function in different ways. One drawback to this vector is that it does not express ADP and thus, probably does not rapidly kill tumor cells by efficient virus release and cell-to-cell spread, whereas certain vectors of the present invention overexpress ADP and efficiently kill infected cells.

While many armed vectors have been constructed, only a few have been examined for enhanced efficacy in tumor models. The transgene usually falls into one of three categories; pro-drug converting enzyme for suicide gene therapy, cytokine gene to augment anti-tumor immune responses, or pro-apoptotic protein or polynucleotide. RC vectors expressing the HSV-TK gene have shown mixed results with regard to suppressing the growth of human xenograft tumors (Lambright et al., 2001; Morris and Wildner, 2000; Wildner and Morris, 2000; Wildner et al., 1999a; Wildner et al., 1999b; Nanda et al., 2001). Other RC vectors with pro-drug converting enzymes have demonstrated enhanced efficacy relative to control vectors that do not express the transgene (Rogulski, 2000; Stubdal et al., 2003). Interferon γ was also able to increase the efficacy of an RC vector (Zhang et al, 1996). Furthermore, a TNF-α-expressing vector was more effective at inhibiting tumor growth than it's control counterpart (Kurihara et al., 2000). RC vectors expressing the proapoptotic protein p53 have been constructed, but these vectors have not yet been tested in animal models (Haviv et al., 2002; Sauthoff et al., 2002; van Beusechem et al., 2002; Koch et al, 2001). The inventors have created RC vectors that express human TRAIL (TNF-related apoptosis-inducing ligand), a tumor selective pro-apoptotic protein (U.S. Provisional Application Ser. No. 60/458,493, which is incorporated herein by reference in its entirety).

As an alternative to directly arming the RC vector, the inventors have developed a multi-vector strategy in which an RD vector provides the transgene while the RC vector supplies the viral genes required for vector replication (Doronin et al., 2000; Doronin et al., 2003; Habib et al., 2002). This approach has several advantages compared to armed RC vectors. First, the RD genome will be amplified, boosting transgene expression, packaged and released at the culmination of infection, enhancing vector spread. Second, because a greater proportion of the Ad genome is deleted in RD vectors, larger and/or additional transgenes can be incorporated into RD vectors than into RC vectors. Third, many RD vectors that express tumor-selective transgenes already exist, eliminating the need to construct RC vectors expressing these same genes. Fourth, more than one RD vector could be used simultaneously, thus allowing expression of more than one transgene, and possibly more than one tumor cell killing mechanism.

The idea of simultaneously infecting xenograft tumors with RC and RD vectors has been tested. Habib et al. showed that two different RC vectors were able to enhance the spread of an RD vectors that expressed a reporter gene (Habib et al, 2002). Co-infection of xenograft tumors with an RC vector and an IL-12-expressing RD vector resulted in increased IL-12 production in the tumor and greater anti-tumor efficacy compared to the RD or RC vector alone (Nagayama et al., 2003). Similar results were obtained with an IL-2-expressing RD vector (Motoi et al., 2000).

Typically, the choice of which transgene to use is important. To avoid damage to normal tissues this effector molecule should be tumor-selective. It would also be beneficial to use a protein that induces significant bystander effect or one that is secreted so that it could diffuse freely away from the infected cell to kill tumor cells at a distance.

Exemplary RD Ad vectors differ only with respect to the transgene that is expressed. These Ad5-based vectors contain a full deletion of the E1 region and a partial deletion of E3 (RIDα, RIDβ, and E314.7K). The expression cassette may comprise a promoter, such as a cytomegalovirus immediate early promoter, driving expression a transgene cDNA (with minimal 5′ or 3′ untranslated sequences) followed by a polyadenylation signal, e.g., a SV40 late polyadenylation signal. Although each of the exemplary therapeutic transgenes (p53, MDA-7, FUS-1, PTEN) is known to function in quite distinct molecular pathways, the overall result of pharmacologic hyper-expression of these tumor suppressor gene products (which are endogenously very tightly regulated) is to cause growth and cell cycle arrest and induce apoptosis in tumor cells. In general, these effects are not observed in normal cells.

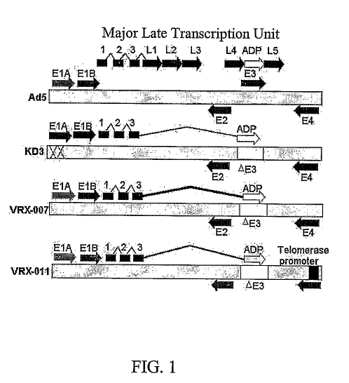

RC oncolytic Ad vectors that overexpress the Adenovirus Death Protein (ADP) have been described (see FIG. 1 and U.S. Pat. No. 6,627,190, which is incorporated herein by reference in its entirety). ADP is required for efficient virus release and cell-to-cell spread at the culmination of the Ad infectious cycle. Overexpression of ADP, as compared to wild-type Ad, enhances spread of adenovirus through cultured cells and enhances efficacy in nude mouse subcutaneous xenograft tumor models.

In order to develop a more potent oncolytic vector, the inventors describe the combination of transgene and vector platform (armed RC vector or multi-vector) that augment the efficacy of ADP overexpressing adenoviruses. Pre-existing RD vectors are available with known anti-cancer efficacy. Embodiments of the invention include one or more armed ADP overexpressing adenoviruses encoding one or more therapeutic polynucleotide, one or more armed ADP overpressing adenoviruses encoding one or more therapeutic polynucleotide in combination with one or more replication deficient adenoviruses encoding one or more therapeutic polynucleotide, or one or more replication competent adenoviruses overexpressing ADP in combination with one or more replication deficient adenoviruses encoding one or more therapeutic polynucleotide.

To enhance the potency of the RC vector described herein, several RC vectors are constructed, each armed with a different transgene that is known to kill or inhibit the growth of cancer cells. Exemplary transgenes include p53, MDA-7, FUS-1 and PTEN, which all have pro-apoptotic activity. Preclinical studies with the corresponding RD vectors (e.g., Ad-p53, Ad-mda7, and Ad-PTEN) show that each vector kills a broad spectrum of cancer cells, and clinical trials with Ad-p53 (Phase I, II and III) and Ad-mda7 (Phase I and Phase II) are ongoing. All three of these exemplary vectors are characterized in vitro with regard to ADP and transgene expression, replication proficiency, and cell killing ability. In certain embodiments the RD vector may overexpress ADP and the RC vector expresses a nucleic acid encoding a therapeutic polypeptide, so that the RC virus may be propagated by ADP being supplied in trans.

Another application of armed adenoviruses, RD and RC is to generate immune responses to target antigens expressed by nucleic acids incorporated into the RD and RC. These antigens may be tumor derived or derived from infectious pathogens or other targets against which an immune response would have benefit in preventing or treating disease. The immune targets are known to those skilled in the art for applications in the treatment or prevention of infectious diseases, cancer and autoimmune diseases. In a preferred embodiment, the RD encodes a gene expressing the target antigen which is mixed with an RC to result in the replication of both viruses enhancing immune responses to the target antigen. A variety of RD and RC may be combined for this purpose that may contain complementary sequences permitting their replication only in cells co-infected with both vectors or in specific types of cells. Safety may also be further increased by lowering the ratio of RC to RD in the combined preparations. RC alone expressing target antigens with adenoviral genes needed for replication under the control of inducible promoters is another approach to manage safety by permitting only transient vector replication.

A. Methods for Producing Viral Particles

The traditional method for the generation of adenoviral particles is co-transfection followed by subsequent in vivo recombination of a shuttle plasmid (usually containing a small subset of the adenoviral genome and the gene of interest in an expression cassette) and an adenoviral helper plasmid (containing most of the entire adenoviral genome) into either 293 or 911 cells (Introgene, The Netherlands). In the present invention, certain adenoviruses are replication-competent so that helper cells expressing adenoviral proteins are not necessarily needed, but may be used as appropriate. After transfection, adenoviral plaques are isolated from the agarose overlaid cells and viral particles are expanded for analysis. For detailed protocols the skilled artisan is referred to Graham and Prevac, 1991.

Alternative technologies for the generation of adenovirus or adenovirus expression vectors include utilization of the bacterial artificial chromosome (BAC) system, in vivo bacterial recombination in a recA+bacterial strain utilizing two plasmids containing complementary adenoviral sequences, and the yeast artificial chromosome (YAC) system (PCT publications 95/27071 and 96/33280, which are incorporated herein by reference).

B. Modifications of Adenovirus

Modifications of RC and RD adenoviruses described herein may be made to improve the ability of the adenovirus to treat cancer or other disease states. The present invention also includes any modification of an adenovirus that improves the ability of the adenoviruses to treat neoplastic cells. Included are modifications to the adenovirus genome in order to enhance the ability of the adenovirus to infect and replicate in cancer cells, e.g., by altering the receptor binding molecules (U.S. Pat. No. 6,555,367 and U.S. Patent Applications 20030175973, 20030175243, 20030143209, 20020192187, and 20020123147, each of which is incorporated herein by reference in its entirety).

The absence or the presence of low levels of the coxsackie virus and adenovirus receptor (CAR) on several tumor types can limit the efficacy of the adenovirus. Various peptide motifs may be added to the fiber knob, for instance an RGD motif (RGD sequences mimic the normal ligands of cell surface integrins), Tat motif, poly-lysine motif, NGR motif, CTT motif, CNGRL motif, CPRECES motif or a strept-tag motif (Ruoslahti and Rajotte, 2000). A motif, such as RGD, can be inserted into the HI loop of the adenovirus fiber protein. Modification of the capsid allows the adenoviral construct to bind to integrins without binding CAR. The motifs allow CAR-independent target cell infection. This allows higher replication, more efficient infection, and increased lysis of some tumor cells (Suzuki et al., 2001, incorporated herein by reference). Peptide sequences that bind specific human glioma receptors such as EGFR or uPR may also be added. Specific receptors found exclusively or preferentially on the surface of cancer cells may also be a target for adenoviral binding and infection, such as EGFRvIII.

Modifications of an oncolytic adenovirus genome also include inserting expression cassettes into the adenovirus genome to express polynucleotides encoding therapeutic polypeptides within tumor cells. Possible therapeutic polynucleotides may encode various therapeutic agents including pro-drug converting enzymes, pro-apoptotic proteins and nucleotides, and cytokines. Anti-angiogenesis molecules such as anti-sense VEGF, dominant negative forms of angiogenesis-related receptors, inhibitors of metalloproteases, or a gene coding for endostatin or angiostatin may also be incorporated into an adenoviral expression vector of the present invention. Still other foreign genes include anti-apoptotic molecules such as Bcl-2 and immunomodulators such as interferon gamma, alpha, or beta, or interleukin molecules.

Modifications may be made to an adenoviral genome in order to increase the ability of the adenovirus to escape from an anti-viral immune response. For example, the negative charge of the adenoviral capsid may be modified.

Regulatory elements may be inserted into an adenoviral genome in order to control temporal expression of adenoviral genes or target certain tissues or cells for adenoviral replication. Regulatory elements that could be inserted include GAFP-related promoters, prednisone-sensitive enhancers, the CEA promoter or E2F gene regulated control elements in conjunction with adenoviral or heterologous genes.

### II. Nucleic Acids

The present invention concerns therapeutic nucleic acids that are capable of expressing a therapeutic polynucleotide, protein, polypeptide, or peptide, such as p53 (GenBank accession AY429684, incorporated herein by reference), MDA-7 (GenBank accession U16261, incorporated herein by reference), PTEN (GenBank accession U93051, incorporated herein by reference), FUS-1 (GenBank accession AF055479) or FHIT (GenBank accession U46922, incorporated herein by reference), particularly, in some instances, the human sequences. A DNA segment encoding a therapeutic polynucleotide and/or polypeptide refers to a DNA segment that contains wild-type, polymorphic, or mutant therapeutic polynucleotide and/or polypeptide-coding sequences that encode a functional protein. It will be understood that in the case of tumor suppressors, the function is tumor suppression, suppression of angiogenesis, or both. In some cases, the function may be the ability to induce apoptosis. Included within the term “DNA segment” are polynucleotides, DNA segments smaller than a polynucleotide, and recombinant vectors. Recombinant vectors may include plasmids, cosmids, phage, viruses, and the like. In certain embodiments recombinant adenoviruses are contemplated. In particular, an adenovirus comprising an expression cassette or polynucleotide encoding a p53, MDA-7, PTEN, FUS-1 or FHIT polypeptide is contemplated.

As used in this application, the term “polynucleotide” refers to a nucleic acid molecule of greater than 3 nucleotides. Therefore, a “polynucleotide encoding a p53, MDA-7, PTEN, FUS1 or FHIT” refers to a DNA segment that encodes p53, MDA-7, PTEN, FUS1, FHIT, or other therapeutic polypeptide or polynucleotide.

Similarly, a polynucleotide comprising an isolated or purified transgene, such as a p53, MDA-7, PTEN, FUS1 or FHIT transgene refers to a DNA segment including p53, MDA-7, PTEN, FUS1 or FHIT polypeptide coding sequences and, in certain aspects, regulatory sequences, isolated substantially away from other naturally occurring genes or protein encoding sequences. In this respect, the term “transgene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants. The nucleic acid encoding a therapeutic polynucleotide, e.g., p53, MDA-7, PTEN, FUS-1 or FHIT may contain a contiguous polynucleotide sequence encoding all or a portion of a therapeutic polypeptide, e.g., p53, MDA-7, PTEN, FUS-1 or FHIT, of the following lengths: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, or more nucleotides or base pairs.

“Isolated substantially away from other coding sequences” means that the transgene of interest, for example the polynucleotide encoding a p53, MDA-7, PTEN, FUS-1 or FHIT polypeptide, forms part of the coding region of the DNA segment, and that the DNA segment does not contain large portions of unrelated naturally-occurring coding DNA. Of course, this refers to the DNA segment as originally isolated, and does not exclude transgenes or coding regions later added to the segment by human manipulation.

The nucleic acid segments used in the present invention, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

The DNA segments used in the present invention may encode biologically functional equivalent therapeutic polypeptides such as p53, MDA-7, PTEN, FUS-1 or FHIT proteins and peptides. Such sequences may arise as a consequence of codon redundancy and functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by human may be introduced through the application of site-directed mutagenesis techniques, e.g., to decrease the antigenicity of the protein or to inhibit binding to a given protein.

In yet another embodiment, multiple adenoviruses and nucleic acids may be involved in a therapy or treatment. For instance, a RD adenovirus armed with a therapeutic polynucleotide may be administered before, during or after a RC adenovirus. Delivery of a second therapeutic polynucleotide in conjunction with a vector encoding one of the following gene products may have a combined therapeutic effect on target tissues. A variety of therapeutic polynucleotides are encompassed within the invention.

A. Therapeutic Polynucleotides

A therapeutic polynucleotide of the invention typically falls into one of three categories; pro-drug converting enzyme for suicide gene therapy, cytokine gene to augment anti-tumor immune responses, or pro-apoptotic protein or polynucleotide. Although, other therapeutic polynucleotides such as anti-sense RNA, miRNA, and siRNA are also contemplated, particularly when the down regulation of a growth promoting gene is desired.

1. Inhibitors of Cellular Proliferation

Tumor suppressors function to inhibit excessive cellular proliferation. The inactivation of these genes destroys their inhibitory activity, resulting in unregulated proliferation. Non-limiting examples of tumor suppressors that may be employed in the compositions and methods of the invention include p53, MDA-7, FHIT, FUS-1 and PTEN.

Other transgenes that may be employed according to the present invention include Rb, p16, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC (mutated in colorectal cancer).

2. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

3. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. However, oncogenes rarely arise from genes encoding growth factors. In one embodiment of the present invention, it is contemplated that anti-sense mRNA or siRNA directed to a particular inducer of cellular proliferation may be used to prevent expression of the inducer of cellular proliferation.

The proteins FMS and ErbA are growth factor receptors, like ErbB. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity. The proteins Jun, Fos and Myc are proteins that directly exert their effects on nuclear functions as transcription factors.

B. Antigens and Vaccines

The present invention also provides vectors, compositions and methods useful for vaccination. The antigen can be presented in the adenovirus capsid, alternatively, the antigen can be expressed from a heterologous nucleic acid introduced into a recombinant adenovirus genome and carried by the inventive adenoviruses, either RD or RC Ads. Any immunogen of interest can be provided by the adenovirus vector.

The antigen expressed by cells infected by adenovirus is processed and displayed in the infected cells in a way that mimics pathogen-infected cells. Further, the recombinant adenovirus may infect dendritic cells which are very potent antigen-presenting cells. Still further, the recombinant adenovirus may also carry genes encoding immuno-enhancing cytokines to further boost immunity. Moreover, the recombinant adenovirus may naturally infect airway and gut epithelial cells in humans, and therefore the vaccine may be delivered through nasal spray or oral ingestion.

In still yet another embodiment, the present invention is directed to both prophylactic and therapeutic immunization. Therapeutic administration of the polynucleotide or polypeptide vaccine to infected subjects is effective to delay or prevent the progression of various infections and disease states, and also to arrest or treat such states. Prophylactic administration of the adenovirus vaccine to subjects is effective to reduce the morbidity and mortality associated with various disease states. Further, if a vaccinated subject is subsequently infected or develops a disease state, the vaccine is effective to prevent progression of the initial infection or disease state. As discussed in more detail hereinbelow, the vaccine can contain or encode a single immunogenic polypeptide or multiple immunogenic polypeptides.

In another embodiment, the invention is directed to therapeutic immunization using a polynucleotide vaccine that preferably stimulates an antibody response, a cell-mediated CD4 (+) immune response and a CD8 (+) immune response. The vaccine is administered to a subject predisposed to or may possible develop a hyperproliferative disorder. It is contemplated that the vaccine can cause the subject to either clear a diseased cell, or at least arrest development of disease, thereby preventing or delaying progression of the disease to a chronic debilitating or life threatening state. For example, the vaccine of the invention is expected to be effective against precancerous, cancerous, or hyperproliferative cells, as well as other disease known to one of skill in the art are attenuated by vaccination. It is to be understood that the polynucleotide encoding an antigen to be used in the vaccination may be derived from a cell or organism against which the vaccine is directed.

The choice of polynucleotide delivery as an immunization technique offers several advantages over other vaccine or antigen delivery systems. Vaccines containing genetic material are favored over traditional vaccines because of the ease of construction and production of the vectors, the potential for modification of the sequences by site-directed mutagenesis to enhance the antigenic potency of the individual epitopes or to abolish epitopes that may trigger unwanted response, in the case of DNA vaccines, the stability of DNA, the lack of the dangers associated with live and attenuated vaccines, their ability to induce both humoral and cell mediated immunity and, in particular, CD8 (+) T cell responses, and the persistence of the immune responses. Representative papers describing the use of DNA vaccines in humans and primates include Endresz et al. (1999); McCluskie et al. (1999); Wang et al. (1998); Le Borgne et al. (1998); Tacket et al (1999); Jones et al. (1999); Wang et al. (1998). The ability to enhance the immune response by the co-delivery of genes encoding cytokines is also well-established.

The Adenoviral vaccine of the invention includes at least one, two, three or more nucleotide coding regions, each coding region encoding an immunogenic polypeptide component. The coding reginos may be in the same or different adenoviral vector, each of which may be a RD or RC Ad. When it contains two or more nucleotide coding regions, the polynucleotide vaccine is referred to herein as a “multicomponent” polynucleotide vaccine.

In addition, the vector construct can contain nucleotide sequences encoding cytokines, such as granulocyte macrophage colony stimulating factor (GM-CSF), interleukin-12 (IL-12) and co-stimulatory molecules such B7-1, B7-2, CD40. The cytokines can be used in various combinations to fine-tune the response of the subject's immune system, including both antibody and cytotoxic T lymphocyte responses, to bring out the specific level of response needed to control or eliminate the infection or disease state. The polynucleotide vaccine can also encode a fusion product containing the antigenic polypeptide and a molecule, such as CTLA-4, that directs the fusion product to antigen-presenting cells inside the host. An alternative approach to delivering the adenoviral polynucleotide to a subject involves the use of a viral or bacterial vector. Examples of suitable viral vectors include polio virus, pox viruses such as vaccinia, canary pox, and fowl pox, herpes viruses, including catfish herpes virus, adenovirus-associated vector, and retroviruses. Exemplary bacterial vectors include attenuated forms of Salmonella, Shigella, Edwardsiella ictaluri, Yersinia ruckerii, and Listeria monocytogenes.

An “immunogenic polypeptide” or “antigen” is a polypeptide derived from the cell or organism that elicits in a subject an antibody-mediated immune response (i.e., a “B cell” response or humoral immunity), a cell-mediated immune response (i.e. a “T cell” response), or a combination thereof. A cell-mediated response can involve the mobilization helper T cells, cytotoxic T-lymphocytes (CTLs), or both. Preferably, an immunogenic polypeptide elicits one or more of an antibody-mediated response, a CD4 (+) Th1-mediated response (Th1: type 1 helper T cell), and a CD8 (+) T cell response. It should be understood that the term “polypeptide” as used herein refers to a polymer of amino acids and does not refer to a specific length of a polymer of amino acids. Thus, for example, the terms peptide, oligopeptide, and protein are included within the definition of polypeptide.

An antigen may be derived from a pathogen or may be a self antigen in the case of a cancer vaccine or other self antigen associated with a non-infectious, non-cancer chronic disorder such as allergy. The vaccine may be a nucleic acid alone or it may also comprise an adjuvant or other stimulant to improve and/or direct the immune response, and may also further comprise pharmaceutically acceptable excipient(s).

Diseases against which a subject may be immunized include viral diseases, allergic manifestations, diseases caused by bacterial or other pathogens, such as parasitic organisms, AIDS, autoimmune diseases such as Systemic Lupus Erythematosus, Alzheimer's disease and cancers. Suitable antigens comprise bacterial, viral, fungal and protozoan antigens derived from pathogenic organisms, as well as allergens, and antigens derived from tumors and self-antigens. Typically, the antigen will be a protein, polypeptide or peptide antigen.

The methods and compositions described herein provide a means for treating a variety of malignant cancers. For example, the system of the present invention can be used to mount both humoral and cell-mediated immune responses to particular proteins specific to the cancer in question, such as an activated oncogene, a fetal antigen, or an activation marker. Such tumor antigens include any of the various MAGEs (melanoma associated antigen E), including MAGE 1, 2, 3, 4, etc. (Boon, 1993); any of the various tyrosinases; MART 1 (melanoma antigen recognized by T cells), mutant ras; mutant p53; p97 melanoma antigen; CEA (carcinoembryonic antigen), among others.

Specific examples of antigens useful in the present invention include a wide variety of proteins from the herpesvirus family, including proteins derived from herpes simplex virus (HSV) types 1 and 2, such as HSV-1 and HSV-2 glycoproteins gB, gD and gH; antigens derived from varicella zoster virus (VZV), Epstein-Barr virus (EBV) and cytomegalovirus (CMV) including CMV gB and gH; and antigens derived from other human herpesviruses such as HHV6 and HHV7. (See, e.g. Chee et al., Cytomegaloviruses (McDougall, 1990) for a review of the protein coding content of cytomegalovirus; McGeoch et al. (1988), for a discussion of the various HSV-1 encoded proteins; U.S. Pat. No. 5,171,568 for a discussion of HSV-1 and HSV-2 gB and gD proteins and the genes encoding therefor; Baer et al. (1984), for the identification of protein coding sequences in an EBV genome; and Davison and Scott (1986), for a review of VZV.)

Antigens derived from other viruses will also find use in the inventive methods, such as without limitation, proteins from members of the families Picornaviridae (e.g., polioviruses, etc.); Caliciviridae; Togaviridae (e.g., rubella virus, dengue virus, etc.); Flaviviridae; Coronaviridae; Reoviridae; Birnaviridae; Rhabodoviridae (e.g., rabies virus, etc.); Filoviridae; Paramyxoviridae (e.g., mumps virus, measles virus, respiratory syncytial virus, etc.); Orthomyxoviridae (e.g., influenza virus types A, B and C, etc.); Bunyaviridae; Arenaviridae; Retroviradae (e.g., HTLV-I; HTLV-II; HIV-1 (also known as HTLV-III, LAV, ARV, hTLR, etc.)), including but not limited to antigens from the isolates HIV (III) b′ HIV (SF2), HIV (LAV), HIV (LAI), HIV (MN)); HIV-1 (CM235), HIV-1 (US4); HIV-2; simian immunodeficiency virus (SIV) among others. See, e.g. Joklik, 1988); Fields and Knipe (1991), for a description of these and other viruses.

Additionally, the envelope glycoproteins HA and NA of influenza A are of particular interest for generating an immune response. Numerous HA subtypes of influenza A have been identified (Kawaoka et al., 1990; Webster et al., 1983. Thus, proteins derived from any of these isolates can also be used in the techniques described herein.

The compositions and methods described herein will also find use with numerous bacterial antigens, such as those derived from organisms that cause diphtheria, cholera, tuberculosis, tetanus, pertussis, meningitis, and other pathogenic states, including, without limitation, Bordetella pertussis, Neisseria meningitides (A, B, C, Y), Hemophilus influenza type B (HIB), and Helicobacter pylori. Examples of parasitic antigens include those derived from organisms causing malaria and Lyme disease.

C. Cloning, Gene Transfer, and Expression

Nucleic acids encoding the adenoviruses and the adenoviruses of the present invention can be constructed using methods known in the art and described herein. Expression of heterologous polynucleotides require that appropriate signals be provided which include various regulatory elements, such as enhancers/promoters that may be derived from both viral and mammalian sources that drive host cell expression of the polynucleotide of interest. Elements designed to optimize messenger RNA stability and translatability in host cells also are defined.

1. Selectable Markers

The markers listed below can be inserted as a heterologous sequence in the genome of one or more adenovirus of the invention, including a RC Ad, RD Ad or both. In certain embodiments of the invention, cells contain a nucleic acid construct of the present invention, a cell may be identified in vitro or in vivo by including a marker in the nucleic acid to be delivered. Such markers may confer an identifiable change to the cell permitting easy identification of cells containing the nucleic acid. The inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also may be employed. The selectable marker employed is not believed to be important, so long as it is capable of being expressed in a cell comprising the nucleic acid of interest. Further examples of selectable markers are well known to one of skill in the art.

D. Promoters and High Level Expression

In one embodiment of the present invention, expression of a tumor suppressor and/or ADP is achieved by placing coding regions for these proteins under the control of the Adenovirus MLP. While providing high level expression of the upstream coding region, the downstream coding region is not expressed as highly. Thus, in accordance with the present invention, various other promoters may be used to drive the expression of the downstream gene (or the second gene that is or is not placed under the control of the MLP). A number of promoter options are available, as discussed below.

One of the goals of the invention is to provide expression of a tumor suppressor and/or overexpression of ADP. Overexpression of ADP, with regard to ADP expression from wild-type Ad5 virus at 24 hours p.i., may be 1.5-fold, 2-fold, 3-fold, 4-fold, 5-fold, 10-fold or greater.

1. Adenovirus Major Late Promoter

At the onset of DNA replication, the pattern of Adenoviral transcription changes radically from the early to the late genes. There is cis-acting control of this switch, i.e., only newly replicated DNA is used for late gene transcription, but the mechanism controlling this is not understood. Late phase transcription is driven primarily by the major late promoter (MLP). Although transcription from this promoter is complex, involving multiple polyadenylation signals and an elaborate usage of RNA splicing, five gene clusters can be defined (L1-L5). Late phase gene expression is primarily concerned with the synthesis of virion proteins. A tripartite leader sequence is found in the 5′ region of the late transcripts. Just upstream of the first splice site is a cap structure, to which 5′GTP is added. Thirty-one base pairs upstream of the promoter is a TATAAAA sequence, but this is not necessary for transcription.

2. Tumor Specific Promoters

Tumor specific promoters may be used in conjunction with an amplifying expression system, described herein. The expression system relies, in the first instance, on the ability of a tissue specific promoter to drive the expression of a transcriptional transactivator, which then turns on a second promoter of interest. In fact, the promoter need not be entirely specific for tumor tissue but, rather, should be active preferentially in tumor tissue. In other words, a small amount of expression in normal tissues, as compared to tumor tissues, may be tolerated. The following tumor specific (or preferential) promoters are contemplated for use in accordance with the present invention.

a. Carcinoembryonic Antigen (CEA) Promoter

CEA is a membrane glycoprotein that is overexpressed in many carcinomas and is widely used as a clinical tumor marker (Paxton et al, 1987; Thompson et al., 1991). Sequence analysis has identified several molecules that are closely related to CEA, including non-specific cross-reacting antigens (NCA) and biliary glycoprotein (Neumaier et al., 1988; Oikawa et al., 1987; Hinoda et al., 1991). CEA is expressed at low levels in some normal tissues and is usually overexpressed in malignant colon cancers and other cancers of epithelial cell origin. Both CEA and NCA expression is fairly homogenous within metastatic tumors, presumably due to the important functional role of these antigens in metastasis (Robbins et al., 1993; Jessup and Thomas, 1989).

The cis-acting sequence that confers expression of the CEA gene on certain cell types has been identified and analyzed (Hauck and Stanners, 1995; Schrewe et al., 1990; Accession Nos. Z21818 and AH003050). It consists of approximately 400 nucleotides upstream from the translational start codon and has sequence homology with a similar sequence in NCA (Schrewe et al., 1990). This promoter has been used to drive some suicide genes and to mediate cell killing in tumor xenografts of stably transfected cells (Osaki et al., 1994; Richards et al., 1995). However, its application in gene therapy is limited by its relatively low transcriptional activity. To solve this problem, Kijima et al. (1999) recently used the Cre/loxP system to enhance transgene expression from the CEA promoter. In their system, a stuffer DNA flanked by a loxP sequence was placed between a transgene and a strong upstream promoter. For coadministration with a second vector expressing a Cre gene driven by a CEA promoter, the stuffer DNA was removed to permit expression of the transgene from its upstream promoter. However, this approach requires rearrangement of vector molecules and is limited by the transcriptional activity of the upstream promoter which could be weak in some cell types.

b. hTERT Promoter

Recently, the human telomerase reverse transcriptase (hTERT) has been cloned by several groups and found to be expressed at high levels in primary tumors and cancer cell lines, but repressed in most somatic tissues (Nakamura et al., 1997; Meyerson et al., 1997; Kilian et al., 1997; Harrington et al., 1997). Data suggest that hTERT is a key determinant of telomerase activity. This includes the finding that hTERT expression is highly correlated with telomerase activity and that ectopic expression of hTERT in telomerase-negative cells is sufficient to reconstitute telomerase activity and extend the life span of normal human cells (Nakamura et al., 1997; Meyerson et al., 1997; Kilian et al., 1997; Harrington et al., 1997; Weinrich et al., 1997; Nakayama et al., 1998; Counter et al., 1998; Bodnar et al., 1998). More recently, it was reported that ectopic expression is required, but not sufficient, for direct tumorigenic conversion of normal human epithelial and fibroblast cells (Hahn et al., 1999).

The promoter region of the hTERT gene also has been cloned (Takakura et al., 1999; Horikawa et al., 1999; Cong et al., 1999; Acession Nos. AB016767 and AF097365). The promoter is high G/C (guanine/cytosine)-rich and lacks both TATA and CAAT boxes, but contains binding sites for several transcription factors, including Myc and Sp1. Deletion analysis of the hTERT promoter identified a core promoter region of about 200 bp upstream of the transcription start site. Transient assays revealed that the core promoter is significantly activated in cancer cell lines but is repressed in normal primary cells.

c. PSA Promoter

Prostate specific antigen (PSA) or KLK3 as it is sometimes called, is a serine protease which is synthesized primarily by both normal prostate epithelium and the vast majority of prostate cancers (Accession No. S81389). The expression of PSA is mainly induced by androgens at the transcriptional level via the androgen receptor (AR). The AR modulates transcription through its interaction with its consensus DNA binding site termed the androgen response element (ARE) (Schuur et al, 1996). The core PSA promoter region exhibits low activity and specificity, but inclusion of the PSA enhancer sequence which contains a putative ARE increases expression, specifically in PSA-positive cells. Expression can be further increased when induced with androgens such as dihydrotestosterone (Latham et al., 2000).

d. AFP Promoter.

Alpha-fetoprotein (AFP) is expressed at high levels in the yolk sac and fetal liver and at low levels in the fetal gut (Accession No. L34019). AFP transcription is dramatically repressed in the liver and gut at birth to levels that are barely detectable by postnatal day 28. This repression is reversible as the AFP gene can be reactivated during liver regeneration and in hepatocellular carcinomas. Previous studies in cultured cells and transgenic mice identified five distinct regions upstream of the AFP gene that control its expression. The promoter and three enhancers functioned as positive regulatory elements, whereas the repressor acted as a negative element. The promoter resides within the 250 bp directly adjacent to exon 1. The repressor, a 600 bp region located between −250 and −850, is required for postnatal AFP repression. Further upstream at −2.5, −5.0 and −6.5 kb are three enhancers termed Enhancer I (EI), EII, and EIII. These three enhancers are active, to varying degrees, in the three tissues where AFP is expressed.

e. Probasin and ARR2PB Promoter

One of the most well-characterized proteins uniquely produced by the prostate and regulated by promoter sequences responding to prostate-specific signals, is the rat probasin protein. Study of the probasin promoter region has identified tissue-specific transcriptional regulation sites, and has yielded a useful promoter sequence for tissue-specific gene expression. The probasin promoter sequence containing bases −426 to +28 of the 5′ untranslated region, has been extensively studied in CAT reporter gene assays (Rennie et al., 1993). Prostate-specific expression in transgenic mouse models using the probasin promoter has been reported (Greenberg et al, 1994). Gene expression levels in these models parallel the sexual maturation of the animals with 70-fold increased gene expression found at the time of puberty (2-6 weeks). The probasin promoter (−426 to +28) has been used to establish the prostate cancer transgenic mouse model that uses the fused probasin promoter-simian virus 40 large T antigen gene for targeted overexpression in the prostate of stable transgenic lines (Greenberg et al., 1995). Thus, this region of the probasin promoter is incorporated into the 3′ LTR U3 region of the RCR vectors thereby providing a replication-competent MoMLV vector targeted by tissue-specific promoter elements.

The probasin promoter confers androgen selectivity over other steroid hormones, and transgenic animal studies have demonstrated that the probasin promoter will target androgen, but not glucocorticoid, regulation in a prostate-specific manner. Previous probasin promoters either targeted low levels of transgene expression or became too large to be conveniently used. Thus, a probasin promoter was designed that would be small, yet target high levels of prostate-specific transgene expression (Andriani et al, 2001). This promoter is ARR2PB which is a derivative of the rat prostate-specific probasin promoter which has been modified to contain two androgen response elements. ARR2PB promoter activity is tightly regulated and highly prostate specific and is responsive to androgens and glucocorticoids.

3. Inducible Promoters

Expression of nucleic acids according to the invention can also be controlled by placing one or more genes under the control of a promoter that is activated by an exogenous inducing agent, such as metals, hormones, antibiotics, and temperature changes.

a. Metallothionein Promoters.

U.S. Pat. No. 4,601,978 describes methods and compositions for controlled expression of genes in mammalian host cells. DNA sequences comprising the human metallothionein II (hMT-II) transcriptional regulatory system, inducible by elevated concentrations of heavy metals and glucocorticoids, includes the promoter region (RNA polymerase recognition and binding sites), the transcriptional initiation sequence (cap site), and the regulatory sequence(s) responsible for inducible transcription. The regulatory system is found on a DNA fragment of fewer than about 500 bp (base pairs) located on the 5′ flanking region of the hMT-II gene upstream of the translational initiation codon. See also U.S. Pat. Nos. 5,089,397 and 6,207,146.

b. Glucocorticoid Promoter.

U.S. Pat. No. 5,512,483 discloses a mammalian expression vector containing a synthetic promoter composed of several high affinity glucocorticoid response elements placed upstream of a minimal promoter TATA region. In transiently transfected HeLa cells in the presence of dexamethasone, one of these promoters was at least 50-fold more efficient than the mouse mammary tumor virus long terminal repeat in expressing bacterial chloramphenicol acetyl-transferase (CAT) activity. When the vector was introduced stably into the HeLa cell genome, CAT activity was induced from 10- to more than 50-fold by dexamethasone in 6 of 8 responsive clones. The levels of both basal and induced expression varied from one clone to the next, probably due to an effect of chromosomal location on promoter activity. When propagated stably in HeLa cells in an Epstein-Barr virus episomal vector, the promoter was greater than 50-fold inducible, and its activity was strictly dependent on the presence of dexamethasone. The promoter when stably propagated in HeLa cells was inducible by progesterone in the presence of a transiently transfected progesterone receptor expression vector. These promoters are widely applicable for the strictly controlled high level expression of target genes in eukaryotic cells that contain either the glucocorticoid or progesterone receptors. See also U.S. Pat. Nos. 5,559,027, 5,559,904, and 5,877,018.

c. Tetracycline Response Promoter.

U.S. Pat. No. 5,464,758 discloses a polynucleotide coding for a transactivator fusion protein comprising the tet repressor and a protein capable of activating transcription in eukaryotes. A second polynucleotide molecule coding for a protein, wherein the polynucleotide is operably linked to a minimal promoter operably linked to at least one tet operator sequence is also disclosed. A method to regulate the expression of a protein coded for by a polynucleotide, by cultivating the eukaryotic cell of the invention in a medium comprising tetracycline or a tetracycline analogue is also disclosed.

U.S. Pat. No. 5,851,796 discloses a tetracycline-regulated system which provides autoregulatory, inducible gene expression in cultured cells and transgenic animals is described. In the autoregulatory plasmid pTet-tTAk, a modified tTA gene called tTAk was placed under the control of Tetp. Tetracycline prevents tTA from binding to Tetp, preventing expression of both tTA and luciferase. This negative feedback cycle ensures that little or no tTA is produced in the presence of tetracycline, thereby reducing or eliminating possible toxic effects. When tetracycline is removed, however, this strategy predicts that tiny amounts of tTA protein (which may result from the leakiness of the minimal promoter), will bind to Tet-op and stimulate expression of the tTAk gene. A positive feedforward loop is initiated which in turn leads to higher levels of expression of tTA and thus, luciferase. Polynucleotide molecules encoding the autoregulatory system, as well as methods of enhancing or decreasing the expression of desired genes, and kits for carrying out these methods are described. See also U.S. Pat. Nos. 5,971,122, 6,133,027 and 6,440,741.

d. Heat Shock Protein (hsp) Promoters

The activation and subsequent repression of heat shock genes in Drosophila has been studied by the introduction of cloned segments into Drosophila cells. In particular, the Drosophila hsp70 gene was fused in phase to the E. coli β-galactosidase structural gene, thus allowing the activity of the hybrid gene to be distinguished from the five resident hsp70 heat shock genes in the recipient Drosophila. Drosophila heat shock genes have also been introduced and their activity studied in a variety of heterologous systems, and, in particular, in monkey COS cells (Pelham, 1982; Mirault et al., 1982; and mouse cells (Corces et al., 1981).

The hybrid hsp70-lacZ gene appeared to be under normal heat shock regulation when integrated into the Drosophila germ line (Lis et al., 1983). Three different sites of integration formed large puffs in response to heat shock. The kinetics of puff formation and regression were exactly the same as those of the 87C locus, the site from which the integrated copy of the hsp70 gene was isolated. The insertion of the 7 kilobase E. coli β-galactosidase DNA fragment into the middle of the hsp70 structural gene appeared to have had no adverse effect on the puffing response. The β-galactosidase activity in the transformants was regulated by heat shock.

Deletion analysis of the Drosophila hsp70 heat shock promoter has identified a sequence upstream from the TATA box which is required for heat shock induction. This sequence contains homology to the analagous sequence in other heat shock genes and a consensus sequence CTxGAAxxTTCxAG has been constructed (Pelham and Bienz, 1982). When synthetic oligonucleotides, whose sequence was based on that of the consensus sequence, were constructed and placed upstream of the TATA box of the herpes virus thymidine kinase gene (tk) (in place of the normal upstream promoter element), then the resultant recombinant genes were heat-inducible both in monkey COS cells and in Xenopus oocytes. The tk itself is not heat inducible and probably no evolutionary pressure has occurred to make it heat inducible, but the facts above indicate that tk can be induced by a heat shock simply by replacing the normal upstream promoter element with a short synthetic sequence which has homology to a heat shock gene promoter.

An inverted repeat sequence upstream of the TATA box is a common feature of many of the heat shock promoters which have been studied (Holmgren et al., 1981). In five of the seven Drosophila promoters, this inverted repeat is centered at the 5′-side of the penultimate A residue of the consensus sequence, but the sequence of the inverted repeat itself is not conserved (Pelham, 1982). In some cases, however, the inverted repeat sequence occurs upstream from the TATA box and the consensus sequence is not present. In these cases, there is no heat inducibility so the presence of the inverted repeat does not substitute for the consensus sequence. See also, U.S. Pat. No. 5,521,284. In contrast, U.S. Pat. No. 6,649,260 discloses a cold-inducible promoter.

e. GAL4 Promoter

U.S. Pat. No. 5,013,652 describes a DNA expression vector which can be used to express many heterologous proteins at ultrahigh expression levels of no less than 1 gram per liter of yeast culture or at least 10% of total yeast cell protein. A hybrid yeast promoter was composed of elements from two naturally-occurring yeast promoters. The transcription initiation site was derived from the MF-alpha-1 gene. An upstream activation site derived from the regulatory region of the yeast GAL1-10 gene was utilized in place of the MF-alpha-1 upstream activation site. Use of the GAL1-10 upstream activation site permits tightly regulated expression of the MF-alpha-1 transcription initiation site by metabolites such as glucose and galactose.

The GAL4 protein, encoded by the GAL4 gene, is a positive regulatory protein for the yeast galactose system. It has been shown that this protein binds to the GALL upstream activation site and is required for high level regulated expression of the GAL1 gene. Since most mammalian cells express no GAL4-like activity, a synthetic GAL4-responsive promoter containing GAL4-binding sites and a TATA box should have no or extremely low basal activity in the absence of a GAL4 transactivator, and high activity in its presence. The GAL4 transcriptional activator derived from yeast, that when fused to a highly acidic portion of the herpes simplex virus protein VP16, is a very potent activator of transcription (Sadowski et al., 1988). Thus, genes that have GAL4 binding sites in their promoter regions, are highly activated by the introduction of the GAL4-VP16 fusion protein. A synthetic promoter composed of a minimal TATA box and five consensus 17-mer GAL4-binding site elements (GAL4/TATA) has also been described.

Another transcriptional activator that could be used in a similar manner is a GAL4-estrogen receptor fusion protein (GAL4-ER), where the GAL4 protein is fused to the hormone binding region of the human estrogen receptor (Braselmann et al., 1993). It is envisioned that the VP16 protein could also be added to this complex to render the complex more potent and less cell type restricted, as compared to GAL4-ER alone. The estrogen receptor targets the estrogen response element and thus can be used as an independent regulator of transcription initiation.

E. Internal Ribosome Binding Sites

When combining multiple open reading frames in a single transcript, it may prove desirable to include an internal ribosome entry site (IRES). IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

F. Other Regulatory Elements

Enhancers are genetic elements that increase transcription from a promoter located at a distant position on the same molecule of DNA. Enhancers are organized much like promoters. That is, they are composed of many individual elements, each of which binds to one or more transcriptional proteins. Enhancer/Promoters include but are not limited to enhancers/promoters of Immunoglobulin Heavy Chain, Immunoglobulin Light Chain, T-Cell Receptor, HLA DQ α and DQ β,β-Interferon, Interleukin-2, Interleukin-2 Receptor, MHC Class II, MHC Class II HLA-DRβ,β-Actin, Muscle Creatine Kinase, Prealbumin (Transthyretin), Elastase I, Metallothionein, Collagenase, Albumin Gene, α-Fetoprotein, γ-Globin, β-Globin, c-fos, c-HA-ras, Insulin, Neural Cell Adhesion Molecule (NCAM), α1-Antitrypsin, H2B (TH2B) Histone, Mouse or Type I Collagen, Glucose-Regulated Proteins (GRP94 and GRP78), Rat Growth Hormone, Human Serum Amyloid A (SAA), Troponin I (TN I), Platelet-Derived Growth Factor, Duchenne Muscular Dystrophy, SV40, Polyoma, Retroviruses, Papilloma Virus, Hepatitis B Virus, Human Immunodeficiency Virus, Cytomegalovirus, or Gibbon Ape Leukemia Virus.

Various element/inducer combinations such as MT II/Phorbol Ester (TPA) or Heavy metals; MMTV (mouse mammary tumor virus)/Glucocorticoids; β-Interferon/poly(rI)X or poly(rc); Adenovirus 5 E2/E1A; c-jun/Phorbol Ester (TPA) or H₂O₂; Collagenase/Phorbol Ester (TPA); Stromelysin/Phorbol Ester (TPA) or IL-1; SV40/Phorbol Ester (TPA); Murine MX Gene/Interferon or Newcastle Disease Virus; GRP78 Gene/A23187; α-2-Macroglobulin/IL-6; Vimentin/Serum; MHC Class I Gene H-2 kB/Interferon; HSP70/E1a or SV40 Large T Antigen; Proliferin/Phorbol Ester-TPA; Tumor Necrosis Factor/PMA; Thyroid Stimulating Hormone α Gene/Thyroid Hormone; or Insulin E Box/Glucose, for example.

The basic distinction between enhancers and promoters is operational. An enhancer region as a whole must be able to stimulate transcription at a distance; this need not be true of a promoter region or its component elements. On the other hand, a promoter must have one or more element that direct initiation of RNA synthesis at a particular site and in a particular orientation, whereas enhancers lack these specificities. Promoters and enhancers are often overlapping and contiguous, often seeming to have a very similar modular organization.

Typically, a polyadenylation signal is included to effect proper polyadenylation of the nucleic acid transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

### III. Methods for Treating and Preventing Hyperproliferative Conditions

The present invention involves the treatment of hyperproliferative cells, as well as preventative or prophylactic administration of compositions of the invention to prevent or impede the formation of pre-cancer, cancer or hyperproliferative lesions in a subject, preferable the subject will be pre-disposed or suspect for development of such conditions. The types of conditions that may be treated include conditions that involve hyperproliferative cells with a defective p53, Rb, or other siganling pathway(s). It is contemplated that a wide variety of tumors may be treated using the methods and compositions of the invention, including gliomas, sarcomas, and tumors of the lung, breast, prostate, and/or brain metastases.

In many contexts, it is not necessary that the cell be killed or induced to undergo cell death or “apoptosis.” Rather, to accomplish a meaningful treatment, all that is required is that the tumor growth be slowed to some degree. It may be that the cell's growth is completely blocked or that some tumor regression is achieved. Clinical terms such as “remission” and “reduction of tumor” burden also are contemplated given their normal usage.

The term “therapeutic benefit” refers to anything that promotes or enhances the well-being of the subject with respect to the medical treatment of his/her condition, which includes treatment of pre-cancer, cancer, and hyperproliferative diseases. A list of nonexhaustive examples of this includes extension of the subject's life by any period of time, decrease or delay in the neoplastic development of the disease, decrease in hyperproliferation, reduction in tumor growth, delay of metastases, reduction in cancer cell or tumor cell proliferation rate, improved ability to swallow, improved ability to sleep, a decrease in pain to the subject that can be attributed to the subject's condition and other quality of life measures.

In certain aspects, an individual may be known to be at increased risk of developing hyperproliferative diseases either because of behavior e.g. smokers or genetic background e.g. Familial Adenopolyposis (Spitz et al., 2005; Garber and Offit, 2005; Brawley and Kramer, 2005; Gotay, 2005).

Administration of the armed adenovector formulations that expose them to the organs at risk for developing hyperproliferative conditions is another embodiment of the invention. The methods for administration of these agents for prevention will be known to those skilled in the art and are similar to those utilized for therapeutic purposes.

A. Adenoviral Therapies

Those of skill in the art are well aware of how to apply adenoviral delivery to in vivo and ex vivo situations. For viral vectors, one generally will prepare a viral vector stock. Depending on the kind of virus and the titer attainable, one will deliver 1 to 100, 10 to 50, 100-1000, or up to 1×10⁴, 1×10⁵, 1×10⁶, 1×10⁷, 1×10⁸, 1×10⁹, 1×10¹⁰, 1×10¹¹, 1×10¹², or 1×10¹³ infectious particles to the patient. Formulation as a pharmaceutically acceptable composition is discussed below.

Various routes are contemplated for various tumor types. The section below on routes contains a non-limiting list of possible routes. Where discrete tumor mass, or solid tumor, may be identified, a variety of direct, local and regional approaches may be taken. For example, the tumor may be directly injected with the adenovirus. A tumor bed may be treated prior to, during or after resection and/or other treatment(s). Following resection or other treatment(s), one generally will deliver the adenovirus by a catheter having access to the tumor or the residual tumor site following surgery. One may utilize the tumor vasculature to introduce the vector into the tumor by injecting a supporting vein or artery. A more distal blood supply route also may be utilized.

The method of treating cancer includes treatment of a tumor as well as treatment of the region near or around the tumor. In this application, the term “residual tumor site” indicates an area that is adjacent to a tumor. This area may include body cavities in which the tumor lies, as well as cells and tissue that are next to the tumor.

B. Pharmaceutical Formulations and Delivery

In certain embodiments of the present invention, methods involving delivery of one or more adenovirus compositions are contemplated. In some embodiments, the method is directed to delivery of one or more adenovirus encoding a therapeutic polynucleotide. Examples of diseases and conditions that may be prevented, ameliorated, or treated with one or more adenovirus compositions of the invention include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, breast cancer, bladder cancer and any other diseases or condition related to a cellular hyperproliferative state.

An “effective amount” of the pharmaceutical composition, generally, is defined as that amount sufficient to detectably and repeatedly to achieve the stated desired result, for example, to ameliorate, reduce, minimize or limit the extent of the disease or its symptoms. More rigorous definitions may apply, including elimination, eradication or cure of disease.

1. Administration

In certain specific embodiments, it is desired to kill cells, inhibit cell growth, inhibit metastasis, decrease tumor or tissue size and otherwise reverse or reduce the malignant phenotype of tumor cells, induce an immune response, or inhibit angiogenesis using the methods and compositions of the present invention. The routes of administration will vary, naturally, with the location and nature of the lesion or site to be targeted, and include, e.g., intradermal, subcutaneous, regional, parenteral, intravenous, intramuscular, intranasal, systemic, and oral administration and formulation.

Direct injection, intratumoral injection, or injection into the tumor vasculature is specifically contemplated for discrete, solid, accessible tumors or other accessible target areas. Local, regional or systemic administration also may be appropriate. For tumors of >4 cm, the volume to be administered will be about 4-10 ml (preferably 10 ml), while for tumors of <4 cm, a volume of about 1-3 ml will be used (preferably 3 ml).

Multiple injections delivered as single dose comprise about 0.1 to about 0.5 ml volumes. The viral particles may advantageously be contacted by administering multiple injections to the tumor or targeted site, spaced at approximately 1 cm intervals.

In the case of surgical intervention, the present invention may be used preoperatively, to render an inoperable tumor subject to resection. Alternatively, the present invention may be used before, during or after the time of surgery, or any combination thereof to treat residual or metastatic disease. For example, a resected tumor bed may be injected or perfused with a formulation comprising one or more adenovirus of the invention; a combination of a RC adenovirus and a RD adenovirus encoding one or more therapeutic polynucleotide(s), or a combination of a armed RC adenovirus and one or more RD adenovirus encoding one or more therapeutic polynucleotide(s). The perfusion may be continued post-resection, for example, by leaving a catheter implanted at the site of the surgery. Periodic post-surgical treatment also is envisioned.

Continuous administration also may be applied where appropriate, for example, where a tumor or other undesired affected area is excised and the tumor bed or targeted site is treated to eliminate residual, microscopic disease. Delivery via syringe or catheter is contemplated. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

Treatment regimens may vary as well, and often depend on tumor type, tumor location, immune condition, target site, disease progression, and health and age of the patient. Obviously, certain types of tumors will require more aggressive treatment, while at the same time, certain patients cannot tolerate more taxing protocols. The clinician will be best suited to make such decisions based on the known efficacy and toxicity (if any) of the therapeutic formulations.

In certain embodiments, the tumor or affected area being treated may not, at least initially, be resectable. Treatments with therapeutic viral constructs may increase the resectability of the tumor due to shrinkage at the margins or by elimination of certain particularly invasive portions. Following treatments, resection may be possible. Additional treatments subsequent to resection will serve to eliminate microscopic residual disease at the tumor or targeted site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may conveniently be described in terms of plaque forming units (pfu) or viral particles for a viral construct. Unit doses range from 10³, 10⁴, 10⁵, 10⁶, 10⁷, 10⁸, 10⁹, 10¹⁰, 10¹¹, 10¹², 10¹³ pfu or viral particles (vp) and higher.

2. Injectable Compositions and Formulations

In some embodiments, the method for the delivery of a composition comprising one or more therapeutic adenovirus is via systemic administration. However, the pharmaceutical compositions disclosed herein may alternatively be administered parenterally, subcutaneously, directly, intratracheally, intravenously, intradermally, intramuscularly, or even intraperitoneally as described in U.S. Pat. Nos. 5,543,158; 5,641,515 and 5,399,363 (each specifically incorporated herein by reference in its entirety).

Injection of adenovirus or other vectors for the delivery of nucleic acid constructs may be by syringe or any other method used for injection of a solution, as long as the vectors can pass through the particular gauge of needle required for injection. A novel needeless injection system has been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media which can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the some methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

C. Combination Treatments

In certain embodiments, the compositions and methods of the present invention involve an armed RC or RD adenovirus, or a combination or an armed or unarmed RC adenovirus with an armed or unarmed RD adenovirus encoding a therapeutic polynucleotide, which in turn may be in combination with other agents or compositions to enhance the effect of the adenoviral compositions or to increase any therapeutic, diagnostic, or prognostic effect for which the composition is being employed. These compositions would be provided in a combined amount effective to achieve the desired effect, for example, the killing or growth inhibition of a cancer cell or the inhibition of angiogenesis. This process may involve contacting the cells with the expression construct and the agent(s) or multiple factor(s) at the same time. This may be achieved by contacting the cell with a single composition or pharmacological formulation that includes two or more agents, or by contacting the cell with two or more distinct compositions or formulations wherein at least one composition includes a RC adenovirus and one or more other compositions includes at least a second therapeutic agent.

In one embodiment of the present invention, it is contemplated that RC adenovirus therapy is used in conjunction with immune therapy intervention, in addition to other pro-apoptotic, anti-angiogenic, anti-cancer, or cell cycle regulating agents. Alternatively, the therapy may precede or follow the other agent treatment by intervals ranging from minutes to weeks. In embodiments where one or more second therapeutic agent and a RC composition are applied separately to a cell, tissue, organ or subject, one would generally ensure that a significant period of time did not expire between the time of each delivery, such that the second agent and RC composition would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, for example an RC adenovirus composition is “A” and a second therapy such as chemotherapy is “B”:

| A/B/A B/A/B B/B/A | | A/B/B A/B/B B/A/A | | A/B/B/B | B/A/B/B |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of the oncolytic adenovirus compositions of the present invention to a patient will follow general protocols for the administration of such compositions, taking into account the toxicity, if any. It is expected that the treatment cycles would be repeated as necessary. It also is contemplated that various standard therapies, as well as surgical intervention, may be applied in combination with the described therapy.

In specific embodiments, it is contemplated that an anti-cancer therapy, such as chemotherapy, radiotherapy, or immunotherapy, is employed in combination with the oncolytic adenovirus therapies, as described herein.

1. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves, proton beam irradiation (U.S. Pat. No. 5,760,395 and U.S. Pat. No. 4,870,287) and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic composition, e.g., a RC adenivirla composition, and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

3. Immunotherapy

In the context of cancer treatment, immunotherapeutics, generally, rely on the use of immune effector cells and molecules (e.g., monoclonal antibodies) to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Another immunotherapy could also be used as part of a combined therapy with a RC adenoviral composition. The general approach for combined therapy is discussed herein. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as MDA-7 has been shown to enhance anti-tumor effects (Ju et al., 2000).

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Preferably, human monoclonal antibodies are employed in passive immunotherapy, as they produce few or no side effects in the patient. However, their application is somewhat limited by their scarcity and have so far only been administered intralesionally. Human monoclonal antibodies to ganglioside antigens have been administered intralesionally to patients suffering from cutaneous recurrent melanoma (Irie and Morton, 1986). Regression was observed in six out of ten patients, following, daily or weekly, intralesional injections. In another study, moderate success was achieved from intralesional injections of two human monoclonal antibodies (Irie et al., 1989).

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993). In melanoma immunotherapy, those patients who elicit high IgM response often survive better than those who elicit no or low IgM antibodies (Morton et al., 1992). IgM antibodies are often transient antibodies and the exception to the rule appears to be anti-ganglioside or anticarbohydrate antibodies.

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated anigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders were few compared to those who did not respond.

4. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well

5. Other Agents

It is contemplated that other agents may be used in combination with the present invention to improve the therapeutic efficacy of treatment. These additional agents include immunomodulatory agents that affect the upregulation of cell surface receptors and GAP junctions, cytostatic and differentiation agents, the inhibition of cell adhesion, and the increase in sensitivity of the hyperproliferative cells to apoptotic inducers or other agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Apo2 ligand (Apo2L, also called TRAIL) is a member of the tumor necrosis factor (TNF) cytokine family. TRAIL activates rapid apoptosis in many types of cancer cells, yet is not toxic to normal cells. TRAIL mRNA occurs in a wide variety of tissues. Most normal cells appear to be resistant to TRAIL's cytotoxic action, suggesting the existence of mechanisms that can protect against apoptosis induction by TRAIL. The first receptor described for TRAIL, called death receptor 4 (DR4), contains a cytoplasmic “death domain”; DR4 transmits the apoptosis signal carried by TRAIL. Additional receptors have been identified that bind to TRAIL. One receptor, called DR5, contains a cytoplasmic death domain and signals apoptosis much like DR4. The DR4 and DR5 mRNAs are expressed in many normal tissues and tumor cell lines. Recently, decoy receptors such as DcR1 and DcR2 have been identified that prevent TRAIL from inducing apoptosis through DR4 and DR5. These decoy receptors thus represent a novel mechanism for regulating sensitivity to a pro-apoptotic cytokine directly at the cell's surface. The preferential expression of these inhibitory receptors in normal tissues suggests that TRAIL may be useful as an anticancer agent that induces apoptosis in cancer cells while sparing normal cells. (Marsters et al, 1999).

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radio frequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Armed RC Ad Vectors

Multi-modal therapeutic regimens should be more efficacious than monotherapies because tumor cells resistant to killing through one pathway might be susceptible to killing through another pathway. To augment the effectiveness of RC Ad vector VRX-007, which kills through lytic infection, a therapeutic nucleic acid is expressed that kills cells through an alternative mechanism. The inventors are identifying and performing preclinical studies with an exemplary vector platform, either VRX-007 “armed” directly with a therapeutic nucleic acid or a “multi” vector platform in which tumors are concurrently treated with VRX-007 and an RD Ad vector expressing a therapeutic nucleic acid. Results from these studies will provide for the preparation of an application for clinical trials.

Construct RC Vectors Armed with Potent Anti-Cancer Transgenes

The studies described include the construction of an armed RC Ad vectors based on VRX-007, evaluation of the expression, replication, and cell killing properties of these vectors in cancer cell lines, and assessing the vector-mediated toxicity in normal primary human cells. The inventors have successfully constructed other “armed” RC vectors with the same strategy used to construct the vectors described herein. In addition, studies such as these are routinely performed by the inventors to evaluate other RC Ad vectors.

Construct VRX-007-p53 and VRX-007-PTEN

The inventors have determined that the preferred arrangement of genes within the E3 region of VRX-007 is to place the transgene 3′ or after (relative to transcription) the ORF for ADP. This design maintains ADP overexpression, which is characteristic of VRX-007, while generally providing for abundant expression of the therapeutic nucleic acid. Several vectors have been constructed based on this strategy, most recently VRX-007-mda7. Two additional vectors are to be constructed, in which the cDNA for p53 or PTEN are placed behind the ADP ORF. Following three rounds of plaque purification, purified stocks are prepared by double banding in CsCl. The inventors have now constructed six different RC vectors using this strategy, including a vector that expresses large amounts of the pro-apoptotic protein TRAIL. The vectors retain the Ad anti-apoptotic protein E1B19K, which should prevent apoptosis induced by a pro-apoptotic nucleic acid. An alternative to making VRX-007-p53 or VRX-007-PTEN is adenoviral construction using 293CrmAE3 cell line to perform the recombination and/or grow the vectors. This cell line stably expresses the poxvirus anti-apoptotic protein CrmA and the anti-apoptotic Ad E3 proteins E3-6.7K, RID, and E3-14.7K, thereby blocking multiple apoptotic pathways.

Characterization of VRX-007-mda7, VRX-007-p53, and VRX-007-PTEN in Cancer Cell Lines

These studies focus on new “armed” RC vectors because Ad-p53, Ad-mda7, and Ad-PTEN (the RD vectors) have already been well characterized in various different cancer cell systems. For each “armed” RC vector, the inventors examine the level of expression of ADP and the transgene, perform single step growth curve analysis to assess vector replication, and determine how well different cancer cell lines are killed.

ADP and transgene expression levels are examined by infecting cells at a high MOI and then preparing cell extracts 0.5, 1, 2, and 3 days post infection. As controls, cells are mock infected or infected with VRX-007 or the RD vector that expresses the therapeutic nucleic acid. Western blot analyses is performed to examine the expression level of ADP, the transgene, late proteins (to assure equal infection), and a cellular marker (to assure equal loading). Late in infection (≧1 day) all three vectors will overexpress ADP and that the transgenes will be expressed at a high level.

To assess the kinetics and level of vector replication, replicate cultures of A549 cells are infected at 10 PFU/cell with VRX-007, VRX-007-p53, VRX-007-mda7, or VRX-007-PTEN. Virus harvested at different times post infection are quantified using a plaque assay on A549 cells.

To examine the capacity of the armed vectors to kill cancer cells, a standard “vector spread” assay is performed in different human cancer cell lines. In this assay, cells in a 48-well plate are mock infected or infected with serial 10-fold dilutions of vector (range 10 PFU/cell to 0.00001 PFU/cell). At different times post infection, cells remaining attached to the plate are stained with crystal violet to determine the extent of vector-mediated killing. This assay normally measures the ability of a vector to kill cells by spreading from cell to cell, but in this case will also measure any transgene-induced killing. The inventors use a panel of different cancer cell lines representative of different cancer types, including more than one cell line for all cancer types. These cell lines also represent different genotypes, allowing the assessment of the effects of mutations in different tumor suppressor genes or oncogenes on the ability of a particular therapeutic nucleic acid to augment VRX-007-mediated killing. Human tumor cell lines to be examined include, A549 (lung carcinoma), H460 (lung large cell carcinoma), HepG2 (hepatoblastoma), Hep3B (hepatocellular carcinoma), PC-3 (prostate carcinoma), LNCaP (prostate carcinoma), DLD-1 (colorectal adenocarcinoma), SW480 (colon adenocarcinoma), MCF-7 (breast adenocarcinoma), and MDA-MB-453 (breast carcinoma). The inventors already know that all of these cell lines support vigorous replication of VRX-007. In addition, with the exception of Hep3B cells for which no data is available, all three RD Ad vectors have been shown to function in the cell lines listed above.

While the assay described above is a clear qualitative indicator of the extent of cell killing, the amount of cell death using a cytotoxicity assay is quantified. Cells are infected as for the vector spread assay described above. At different days post infection (p.i.), the cell medium is harvested and, as an indicator of cell death, the amount of lactate dehydrogenase (LDH) activity is determined using a commercially available kit.

Assessment of Toxicity in Cultured Normal Cells Infected with the “Armed” RC Vectors.

Ad-p53, Ad-mda7, and Ad-PTEN are RD vectors so they typically do not replicate in any cells, including normal human cells. Furthermore, extensive testing with Ad-p53 and Ad-mda7 show that these transgenes do not induce apoptosis in cultures of normal cells, despite having that effect on malignant cells. Likewise, the PTEN transgene does not appear to cause apoptosis in the limited number of normal human cell types that have been examined thus far. These data indicate that transgene expression in normal cells should result in little if any toxicity. However, VRX-007 replication is not genetically restricted to cancer cells, indicating that this vector should grow to some extent in normal cells. Therefore, it is important to examine VRX-007-mediated toxicity in normal cells. In addition, it is important to determine if expression of any of the transgenes in the context of a productive Ad infection will result in transgene-induced toxicity. Therefore, the inventors will perform a vector spread assay (described above) in cultures of normal human cells such as human foreskin fibroblasts (HFF), human umbilical vein endothelial cells (HUVEC), human small airway epithelial cells (SAEC), WI-38 cells (fibroblasts), and HEL299 cells (fibroblasts). The toxicity is measured in this assay using both the crystal violet staining technique and the LDH assay.

Employment Tumor Models to Evaluate the Armed vs. Multi-Vector Platforms.

The combination of therapeutic nucleic acid and vector platform (“armed” RC vector or co-infection with VRX-007 and an RD vector) will be assessed to determine which combination is most efficacious in suppressing the growth of tumors in animal model systems. To make this determination, vectors will studied in two different models, the standard nude mouse human tumor subcutaneous xenograft model and a new immunocompetent cotton rat subcutaneous syngeneic tumor model.

Suppression of tumor growth in animal models is a preferred test for any oncolytic agent. However, a suitable model is difficult to find for human Ads because they do not replicate efficiently in other species. The nude mouse human tumor xenograft is the currently accepted model for studying RC Ad vectors, a model with which the inventors have extensive experience. The vectors are to be examined in the recently devised immunocompetent cotton rat model, which will be contingent upon the ability of the different transgenes to function in this animal species. This model should allow the assessment of the effects of the immune system on vector-mediated tumor destruction and on immune-mediated damage to surrounding normal tissue.

At the termination of each study, animals will be necropsied. Tumor, spleen, kidneys, heart, lungs, brain, and liver will be macroscopically examined for signs of pathology and sections of tumor, spleen, lung, and liver will be subjected to histopathological examination, immunohistochemistry to look for expression of Ad late proteins and the transgene, and TUNEL assay to identify cells undergoing transgene-induced apoptosis. An alternative to this analysis is to analyze Ad and transgene expression using real-time PCR assays.

Assessment of Vectors in the Nude Mouse Subcutaneous Tumor Model.

The first choice of cell line is the human Hep3B liver cancer cells. Hep3B tumors are difficult to cure and thus provide a challenging model for assessing the growth suppressing properties of the vectors. An alternative cell line may be used if the in vitro studies demonstrate that liver cancer is not a suitable indication for a particular therapeutic nucleic. Possible alternatives include A549 (lung), LNCaP (prostate), or DLD-1 (colorectal). In a typical experiment, about 5×10⁶ to 1×10⁷ cancer cells are mixed with Matrigel and injected subcutaneously into each hind flank of a nude mouse. When the tumors reach a volume of 50-150 μl, 18 tumors (nine mice) will be injected in four quadrants with vector or vehicle. Tumor volume is measured with a digital caliper three times per week until mice bearing the vehicle-injected tumors require euthanasia (typically about 4 weeks for Hep3B tumors). Any mice that appear to have been cured are maintained for up to 8 weeks to determine if the tumors reappear. Euthanized animals are necropsied and analyzed as described above. Data from this study is used to pick one or more platforms (“armed” or “multi”) to evaluate in the cotton rat tumor model.

In order to assess each transgene in both the “multi” and “armed” vector platform the following treatment groups are assessed in the nude mouse: vehicle, each RD vector alone (Ad-p53, Ad-mda7, Ad-PTEN, and Ad-null [a replication defective vector expressing no transgene]), VRX-007 alone, VRX-007 in combination with each RD vector (VRX-007+Ad-p53, VRX-007+Ad-mda7, VRX-007+Ad-PTEN, and VRX-007+Ad-null), and each “armed” RC vector (VRX-007-p53, VRX-007-mda7, and VRX-007-PTEN) (a total of 13 groups).

Examination of the Ability of Each Transgene to Function in Cotton Rat Cells

To utilize immunocompetent tumor model in which cotton rat tumor cells are grown subcutaneously in cotton rats it must first determine if the p53, MDA-7 and PTEN are able to function in cotton rat cells. The inventors have demonstrated that Ad-p53, Ad-mda7 and Ad-PTEN kill murine tumor cells (unpublished data). Furthermore, the inventors have already shown that wild-type Ad5 and VRX-007 replicate in and kill the LCRT cotton rat tumor cell line. LCRT cells are infected with Ad-p53, Ad-mda7, and Ad-PTEN at 1,000-10,000 viral particles (vp) per cell, a dose that is typically used in human cell lines. At various days post infection cells are stained using the TUNEL assay and then analyzed by flow cytometry. The extent of apoptosis induction is compared to a human cell line infected at the same time and at the same MOI (e.g., conditions have been established for apoptosis induction in A549, DLD-1 and LNCaP lines by all three RD vectors). As controls, cells are mock infected or infected with Ad-null. Those nucleic acids capable of inducing apoptosis in LCRT cells are analyzed further.

Assessment of the Vectors in Subcutaneous Tumors Grown in Immunocompetent Cotton Rats.

As described in the Preliminary Results section, a tumor model has been developed in immunocompetent cotton rats in order to investigate the role of the immune system on vector efficacy and damage to normal tissue surrounding the tumor. This tumor model is used to examine the vectors from the platform that was chosen from the nude mouse studies. If a transgene was shown not to work in LCRT cells then it may not be evaluated in the corresponding vectors in this model. Subcutaneous tumors are established by injecting 1×10⁷ LCRT cells into one flank (up to 15 cotton rats per treatment group). Beginning immediately following injection of the cells, vehicle or vector(s) are administered into the same injection site on four consecutive days. Monitoring of tumor growth and analysis of cotton rat tissues following necropsy is performed as described above. An alternative to the cotton rat immunocompetent tumor model is the Syrian hamster tumor model. (The inventors have shown that VRX-007 and Ad5 replicate quite well, to about the same extent as they do in cotton rat LCRT cells, in three different hamster cancer cell lines [unpublished]. The inventors have also shown that these cell lines form subcutaneous tumors, and, in a preliminary experiment, have shown that VRX-007 suppresses the growth tumors derived from two of these cell lines.)

The outcome the studies will determine which transgene and which vector platform will be used in the toxicology and biodistribution studies and in Phase I clinical trials. If no single therapeutic nucleic acid augments the efficacy of VRX-007 more than the other two, the transgene which functions in the broadest spectrum of cancer cells will be choosen.

### Example 2

### Preclinical Testing

GLP quality vector(s) can be used to perform toxicity and biodistribution studies, which are required for submission of an IND application.

Manufacture of Vector(s).

Introgen therapeutics Inc. has the expertise and facilities to manufacture large batches of research grade RD and RC Ad vectors. They have produced Ad vectors encoding many different transgenes (e.g., human p53, murine p53, MDA-7, PTEN, p16, CCAM, survivin, COX-1, TFPI, etc.) that have been used in GLP toxicology studies and Phase I, II, and III clinical trials in humans, thus the inventors do not anticipate any problems in manufacturing enough vector to be used in the studies. Note that if the “multi” vector platform is chosen two or more vectors, VRX-007 and the RD vectors, will need to be manufactured. Once the vector stock(s) is (are) manufactured the inventors will proceed with biodistribution and toxicology studies.

Perform the Biodistribution and Toxicology Studies.

The toxicology and biodistribution studies are modeled on those that the inventors are currently planning for VRX-007 as a monotherapy. The FDA has recommended that inventors perform toxicity and biodistribution studies for VRX-007 in the cotton rat or Syrian hamster because these immunocompetent animal models should allow the investigation of possible immune-mediated pathology in an animal that is semi-permissive for Ad replication. The inventors anticipate that the cotton rat or Syrian hamster will also be the model of choice for toxicology and biodistribution studies because RC vectors will also be used, however, the FDA will be consulted before these studies are performed. These studies will be performed at Saint Louis University using documentation and control systems modeled after GLP guidelines, or at a contract research organization.

Although the precise protocols will be determined once vector platform is established the basic outlines are described below. If the “armed” vector platform is chosen then a single vector will be used, but the “multi” vector platform will require the use of two vectors simultaneously. The inventors plan to perform an acute toxicology study in non-tumor bearing cotton rats or Syrian hamsters using a single or repeated intravenous dose of vector(s). Three dose groups are to be included (four animals of both sexes for each dose group): 2×10¹² virus particles vp/kg body weight, which is near the maximum tolerated dose of VRX-007 in cotton rats and Syrian hamster, and doses at one and two logs less than the highest dose. Food consumption, weight, and clinical signs are monitored at daily intervals. Animals are sacrificed on days 4 and 29, at which times gross pathology is noted and samples collected for histopathological, hematological, and clinical chemistry assays. The liver, lung, spleen, lymph nodes, brain, heart, kidney, bone marrow, muscle, and gonads are harvested for histopathological analysis. Tissue samples for histopathology are preserved in formalin prior to being embedded in paraffin. Hematological measurements on whole blood include: white, red, and platelet counts, differential counts, hemoglobin, and hematocrit. Clinical chemistry analyses on serum samples include: creatine, total urea nitrogen, alanine aminotransferase, aspartate aminotransferase, alkaline phosphatase, calcium, cholesterol, glucose, total bilirubin, total protein, chloride, sodium, and potassium. If appropriate, serum MDA-7 levels are measured by ELISA.

Biodistribution studies are performed on tumor-bearing cotton rats or Syrian hamsters injected with a single or repeated dose of vector(s). A single LCRT tumor per cotton rat (or HaK tumor per Syrian hamster) is established as described previously. On day 0, tumors are injected with 3×10¹⁰ vp/kg (a dose that should be close to the maximum anticipated dose for the clinical trial). Daily monitoring is performed as for the toxicity study. Animals are sacrificed on days 1 (to establish a baseline before vector replication begins), 4, 10, and 15 (four animals of each sex per time point). Because LCRT tumors are very aggressive, the animals are probably not going to be maintain beyond 15 days. With the HaK Syrian hamster model, the animals may be maintained for up to 30 days, and sacrifice times adjusted accordingly. Upon sacrifice, samples of blood, liver, lung, spleen, brain, heart, gonads, lymph nodes (particularly those near the tumor), tumor, and normal tissue around the tumor are harvested, each with a fresh forceps and scalpel, then flash frozen in liquid nitrogen for subsequent analysis. Genomic DNA is purified from homogenized tissue samples and assayed by quantitative real-time PCR analysis of viral DNA. A CPE assay on tissue homogenates may also be performed to determine the amount of infectious RC vector.

Safety issues.

The inventors chose to develop a VRX-007-based vector for cancer therapy because, to their knowledge, VRX-007 is the most efficacious vector in the human tumor xenograft model described in the public domain to date. This is probably attributable to the fact that VRX-007 spreads more rapidly than Ad5 in nearly all cancer cell lines tested so far (Doronin et al., 2003). Several points bear noting with respect to concerns about the safety of VRX-007-based vectors. First, VRX-007 and its derivatives contain a deletion within the E3 region that should act as a safety feature. This deletion should help prevent runaway vector replication since the E3 proteins primarily function to protect infected cells from attack by the immune system (Lichtenstein et al., 2003). Second, as an additional safety feature, the inventors propose that if a VRX-007-based vector were to be used in humans the vector may be directed to the tumor by intratumoral injection. Third, proposed preclinical studies will address concerns related to undue toxicity mediated by VRX-007 in normal cells. Fourth, possible damage to normal tissues surrounding the tumor will be examined in two different animal models. Particularly important is the study in cotton rats or Syrian hamsters because these species are both semi-permissive for Ad replication (at least in the lungs) and immunocompetent. Fifth, the results of an acute toxicity study with VRX-007 in C57BL/6 mice indicates that VRX-007 is tolerated at up to 1.5×10¹² vp/kg, a level that is very similar to that reported by Onyx Pharmaceuticals for the attenuated mutant ONYX-015 (Heise et al., 1999). In a study by the Novartis group, (Jakubczak et al, 2003) the Ad5 mutant d11520 (which is identical to ONYX-015) produced about the same toxicity at a dose of 6.25×10¹¹ vp/kg in SCID mice as did 1.5×10¹² vp/kg of VRX-007. Interestingly, they also reported that the Ad5 mutant d1327, which lacks most of the E3 region, including the adp gene, caused more toxicity in both SCID mice and C57BL/6 mice at a dose of 6.25×10¹¹ vp/kg than did 1.5×10¹² vp/kg of VRX-007 in the inventors studies. Although they are very similar, VRX-007 overexpresses ADP whereas d1327 does not express any ADP. These considerations indicate that VRX-007 is not unusually toxic, in the C57BL/6 mouse.

Alternatively, instead of VRX-007, KD3 may be used as a the vector backbone (Doronin et al., 2000; Habib et al, 2002) or VRX-011, two conditionally replicating vectors. KD3 differs from VRX-007 only by the presence of two deletions within the E1A gene; one is located within conserved region 2 (CR2) and prevents E1A binding to pRB, while the other deletes a portion of the N-terminal region of E1A and eliminates binding to p300 (Fattaey et al., 1993; Ikeda and Nevins, 1993). Consequently, KD3 selectively replicates in cells with a deregulated cell cycle, namely cancer cells. Replication of VRX-011 is restricted to cells in which the promoter for the human telomerase reverse transcriptase (hTERT) is active because this promoter replaces that of the Ad E4 region, a group of viral genes whose expression is essential for viral replication. The inventors know that VRX-011 grows as well as VRX-007 in LCRT cells so the cotton rat would be a relevant model for this vector as well.

All of the compositions and methods disclosed and claimed herein can be made and executed without undue experimentation in light of the present disclosure. While the compositions and methods of this invention have been described in terms of preferred embodiments, it are apparent to those of skill in the art that variations maybe applied to the compositions and methods and in the steps or in the sequence of steps of the methods described herein without departing from the concept, spirit and scope of the invention. More specifically, it are apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,870,287
U.S. Pat. No. 5,013,652
U.S. Pat. No. 5,089,397
U.S. Pat. No. 5,171,568
U.S. Pat. No. 5,399,363
U.S. Pat. No. 5,464,758
U.S. Pat. No. 5,466,468
U.S. Pat. No. 5,512,483
U.S. Pat. No. 5,521,284
U.S. Pat. No. 5,543,158
U.S. Pat. No. 5,559,027
U.S. Pat. No. 5,559,904
U.S. Pat. No. 5,641,515
U.S. Pat. No. 5,760,395
U.S. Pat. No. 5,846,225
U.S. Pat. No. 5,846,233
U.S. Pat. No. 5,851,796
U.S. Pat. No. 5,877,018
U.S. Pat. No. 5,925,565
U.S. Pat. No. 5,935,819
U.S. Pat. No. 5,971,122
U.S. Pat. No. 6,133,027
U.S. Pat. No. 6,207,146
U.S. Pat. No. 6,440,741
U.S. Pat. No. 6,555,367
U.S. Pat. No. 6,627,190
U.S. Pat. No. 6,649,260
U.S. Prov. Appln. 60/458,493
U.S. Patent Appln. 20020123147
U.S. Patent Appln. 20020192187
U.S. Patent Appln. 20030143209
U.S. Patent Appln. 20030175243
U.S. Patent Appln. 20030175973
Andriani et al., J. Natl. Cancer Inst., 93:1314-1324, 2001.
Baer et al., Nature, 310:207-211, 1984.
Bakhshi et al., Cell, 41(3):899-906, 1985.
Barker and Berk, Virology, 156:107-121, 1987.
Bauzon et al., Mol. Ther., 7:526-534, 2003.
Bodnar et al., Science, 279:349-352, 1998.
Boon, Scientific American, 82-89, 1993.
Braselmann et al., Proc. Natl. Acad. Sci. USA, 90(5):1657-1661, 1993.
Brawley and Kramer, J Clin Oncol 23: 293-300, 2005.
Cleary and Sklar, Proc. Natl. Acad. Sci. USA, (21):7439-7443, 1985.
Cleary et al., J. Exp. Med., 164(1):315-320, 1986.
Cong et al., Hum. Mol. Genet., 8:137-142, 1999.
Corces et al., Proc. Natl. Acad. Sci. USA, 78(11):7038-7042, 1981.
Counter et al., Oncogene, 16:1217-1222, 1998.
Crystal, Cancer Chemo. Pharm., 43 Suppl:S90-S99, 1999.
Davison and Scott, J. Gen. Virol., 67:1759-1816, 1986.
DeWeese et al., 6th Ann. Mtg American Soc. Gene Therapy, 2003.
DeWeese et al., Cancer Res., 61:7464-7472, 2001.
Doronin et al., J. Virol., 74:6147-6155, 2000.
Doronin et al., J. Virol., 75:3314-3324, 2001.
Doronin et al., Virology, 305:378-387, 2003.
Endresz et al., Vaccine, 17:50-58, 1999.
Fattaey et al., Mol. Cell. Biol., 13:7267-7277, 1993.
Freytag et al., Cancer Res., 62:4968-4976, 2002.
Freytag et al., Human Gene Ther., 9:1323-1333, 1998.
Garber and Offit, J Clin Oncol 23: 276-292, 2005.
Gotay, J Clin Oncol 23: 301-310, 2005.
Graham and Prevec, In: Methods in Molecular Biology: Gene Transfer and Expression Protocol, Murray (Ed.), Humana Press, Clifton, N.J., 7:109-128, 1991.
Greenberg et al., Mol. Endocrinol., 8(2):230-239, 1994.
Greenberg et al., Proc. Natl. Acad. Sci. USA, 92(8):3439-3443, 1995.
Habib et al., Cancer Gene Ther., 9:651-654, 2002.
Hahn et al, Nature, 400:464-468, 1999.
Harrington et al., Gene Dev., 11:3109-3115, 1997.
Hauck and Stanners, J. Biol. Chem., 270:3602-3610, 1995.
Haviv et al, Mol. Cancer. Ther., 1:321-328, 2002.
Hawkins and Hermiston, Gene Ther., 8:1142-1148, 2001.
Hawkins et al., Gene Ther., 8:1123-1131. 2001.
Heise et al., Cancer Res., 59:2623-2628, 1999.
Hermiston and Kuhn, Cancer Gene Therapy, 9:1022-1035, 2002.
Hinoda et al., Japanese J. Clin. Oncol., 21:75-81, 1991.
Holmgren et al, Proc. Natl. Acad. Sci. USA, 78(6):3775-3778, 1981.
Horikawa et al, Cancer Res., 59:826-830, 1999.
Ikeda. and Nevins, Mol Cell Biol., 13:7029-7035, 1993.
Irie and Morton, Proc. Natl. Acad. Sci. USA, 83(22):8694-8698, 1986.
Irie et al., Lancet., 1(8641):786-787, 1989.
Jakubczak et al., Cancer Res., 63:1490-1499, 2003.
Jessup and Thomas, Cancer and Metastasis Rev, 8:263-280, 1989.
Jones et al, Vaccine, 17:3065-3071, 1999.
Ju et al., J. Neuropathol. Exp. Neurol., 59(3):241-50, 2000.
Kawaoka et al., Virology, 179:759-767, 1990.
Kerr et al., Br. J. Cancer, 26(4):239-257, 1972.
Kijima et al., Cancer Res., 59(19):4906-4911, 1999.
Kilian et al., Hum. Mol. Genet., 12:2011-2019, 1997.
Koch et al, Cancer Res., 61:5941-5947, 2001.
Kurihara et al, J. Clin. Invest., 106:763-771, 2000.
Lambright et al., Gene Ther., 8:946-953, 2001.
Latham et al, Cancer Res. 60(2):334-41, 2000.
Le Borgne et al., Virology, 240:304-315, 1998.
Lichtenstein et al., Int. Rev. Immunol, 23(1-2):75-111, 2004
Lis et al., Cell, 35(2 Pt 1):403-410, 1983.
Macejak and Sarnow, Nature, 353:90-94, 1991.
Marsters et al., Recent Prog. Horm. Res., 54:225-234, 1999.
McCluskie et al., Mol. Med., 5:287-300, 1999.
McDougall, In: protein coding content of cytomegalovirus, Springer-Verlag, 125-169, 1990.
McGeoch et al., J. Gen. Virol., 69:1531-1574, 1988.
Meyerson et al., Cell, 90:785-795, 1997.
Mirault et al, EMBO J., 1(10): 1279-1285, 1982.
Mitchell et al, Ann. NY Acad. Sci., 690:153-166, 1993.
Mitchell et al., J. Clin. Oncol., 8(5):856-869, 1990.
Morris and Wildner, Mol. Ther., 1:56-62, 2000.
Morton et al., Arch. Surg., 127:392-399, 1992.
Motoi et al., Human Gene Ther., 11:223-235, 2000.
Nagayama et al., Gene Ther., 10:1400-1403, 2003.
Nakamura et al., Science, 277:955-959, 1997.
Nakayama et al., Nature Genet., 18:65-68, 1998.
Nanda et al., Cancer Res., 61:8743-8750, 2001.
Neumaier et al., J. Biol. Chem., 263:3202-3207, 1988.
Oikawa et al. Biochem. Biophys. Res. Comm., 146:464-469, 1987.
Osaki et al., Cancer Res., 54:5258-5261, 1994.
Paxton et al., Proc. Natl. Acad. Sci. USA, 84:920-924, 1987.
PCT Appln. WO95/27071
PCT Appln. WO96/33280
Pelham and Bienz, EMBO J, 1(11): 1473-1477,1982
Pelham, Cell, 30(2):517-528, 1982.
Pelletier and Sonenberg, Nature, 334(6180):320-325, 1988.
Ramachandra et al., Nature Biotech., 19:1035-1041, 2001.
Ravindranath and Morton, Intern. Rev. Immunol, 7: 303-329, 1991.
Remington's Pharmaceutical Sciences, 15^{th} ed., pages 1035-1038 and 1570-1580, Mack Publishing Company, Easton, Pa., 1980.
Rennie et al, Molecular Endocrinology, 7:23-36, 1993.
Richards et al., Hum. Gene Ther., 6:881-893, 1995.
Ring, J. Gen. Virol., 83:491-502, 2002.
Robbins et al, Int. J. Cancer, 53:892-897, 1993.
Rodriguez et al., Cancer Res., 57:2559-2563, 1997.
Rogulski et al., Human Gene Ther., 11:67-76, 2000.
Rosenberg et al., Ann. Surg. 210(4):474-548, 1989.
Rosenberg et al, N. Engl. J. Med., 319:1676, 1988.
Ruoslahti and Rajotte, Annu Rev Immunol., 18:813-27, 2000
Sadowski et al., Nature, 335(6190):563-564, 1988
Sauthoff et al, Hum. Gene Ther., 13:1859-1871, 2002.
Scaria et al., Virology, 191:743-753, 1992.
Schrewe et al., Mol. Cell. Biol., 10:2738-2748, 1990.
Schuur et al., J. Biol. Chem., 271(12):7043-7051, 1996.
Spitz et al, J Clin Oncol 23: 267-275, 2005.
Stubdal et al., Cancer Res., 63:6900-6908, 2003.
Suzuki et al., Clin. Cancer Res, 8:3348-3359, 2002.
Suzuki et al., Clin. Cancer Res., 7:120-126, 2001.
Tacket et al., Vaccine, 17:2826-2829, 1999.
Takakura et al., Cancer Res., 59:551-557, 1999.
Thompson et al., J Clin. Lab. Anal., 5:344-366, 1991.
Tollefson et al., J. Virol., 66:3633-3642, 1992.
Tollefson et al., J. Virol., 70:2296-2306, 1996a.
Tollefson et al., J. Virol., 77:7764-7778, 2003.
Tollefson et al., Virology, 220:152-162, 1996b.
Toth et al., Cancer Gene Ther., 10:193-200, 2003.
Tsujimoto and Croce, Proc. Natl. Acad. Sci. USA, 83(14):5214-5218, 1986.
Tsujimoto et al., Science, 228(4706):1440-1443, 1985.
van Beusechem et al., Cancer Res., 62:6165-6171, 2002.
Virology, 3rd Edition (W. K. Joklik ed. 1988); Fundamental Virology, 2nd Edition (B. N. Fields and D. M. Knipe, eds. 1991
Wang et al., Infect. Immun., 66:4193-202, 1998.
Wang et al., Science, 282(5388):476-480, 1998.
Webster et al., In: Genetics of influenza viruses, Palese and Kingsbury (Eds.), Springer-Verlag, NY, 127-168, 1983.
Weinrich et al., Nature Genet., 17:498-502, 1997.
Wildner and Morris, Cancer Res., 60:4167-4174, 2000a.
Wildner and Morris. J Gene Med., 2:353-360, 2000a.
Wildner et al., Cancer Res., 59:410-413, 1999a.
Wildner et al., Gene Ther, 6:57-62, 1999b.
Wold et al., J. Virol. 52:307-313, 1984.
Ying and Wold, Virology, 313(1):224-234, 2003.
Yu et al., Cancer Res. 59:4200-4203, 1999.
Zhang et al., Proc. Natl. Acad. Sci. USA, 93:4513-4518, 1996.

1. An oncolytic adenovirus comprising an adenovirus death protein gene that is overexpressed in an infected cell, and a nucleic acid encoding a tumor suppressor, wherein all or part of an E3 region is deleted.

2. The oncolytic adenovirus of claim 1, wherein the tumor suppressor is p53, FHIT, MDA7, or PTEN.

3. The oncolytic adenovirus of claim 2, wherein the tumor suppressor is p53.

4. The oncolytic adenovirus of claim 1, wherein the nucleic acid encoding a tumor suppressor is under the control of a heterologous promoter.

5. The oncolytic adenovirus of claim 1, wherein the nucleic acid encoding a tumor suppressor is under the control of an adenoviral MLP promoter.

6. The oncolytic adenovirus of claim 4, wherein the promoter is a constitutive or an inducible promoter.

7. The oncolytic adenovirus of claim 4, wherein the promoter is a CMV IE, dectin-1, dectin-2, human CD11c, F4/80, SM22a MHC class II promoter, SV40, polyoma or adenovirus 2 promoter.

8. An adenoviral composition comprising:
(a) a first replication competent adenovirus comprising
(i) an adenoviral death protein gene that is overexpressed in infected cells, and
(ii) a first nucleic acid encoding a therapeutic protein, wherein all or part of E3 is deleted; and
(b) a second adenovirus comprising a second nucleic acid encoding a therapeutic protein.

9. The composition of claim 8, wherein the first nucleic acid encodes a tumor suppressor.

10. The composition of claim 9, wherein the tumor suppressor is p53, FHIT, MDA7, or PTEN.

11. The composition of claim 10, wherein the tumor suppressor is MDA7.

12. The composition of claim 8, wherein the second nucleic acid encodes a tumor suppressor.

13. The composition of claim 12, wherein the tumor suppressor is p53, FHIT, MDA7, or PTEN.

14. The composition of claim 13, wherein the tumor suppressor is p53.

15. The composition of claim 8, wherein the first nucleic acid and the second nucleic acid encode different therapeutic proteins.

16. The composition of claim 8, wherein the second adenovirus encodes a replication defective adenovirus.

17. The composition of claim 8, wherein the second adenovirus encodes a conditionally replicating adenovirus.

18. The composition of claim 8, wherein the composition is a pharmaceutically acceptable composition.

19. The composition of claim 8, further comprising protamine.

20-27. (canceled)

28. A method of treating a patient with a hyperproliferative disorder comprising administering to a patient an effective amount of an oncolytic adenovirus of claim 1.

29.-37. (canceled)

38. The method of claim 28, wherein the oncolytic adenovirus is administered by injection, perfusion, inhalation or topical application.

39. The method of claim 28, wherein the administration occurs more than once.

40. (canceled)

41. The method of claim 28, further comprising administering to the patient a second therapy, wherein the second therapy is chemotherapy, immunotherapy, surgery, radiotherapy, immunosuppressive agents, or a second gene therapy.

42. The method of claim 41, wherein the second therapy is a second gene therapy.

43. The method of claim 41, wherein the second gene therapy comprises administration of an effective amount of a replication defective adenovirus.

44. The method of claim 41, wherein the second therapy is administered to the patient before administration of the composition comprising the oncolytic adenovirus.

45. The method of claim 41, wherein the second therapy is administered to the patient at the same time as administration of the composition comprising the oncolytic adenovirus.

46. (canceled)

47. The method of claim 41, wherein the chemotherapy comprises an alkylating agent, mitotic inhibitor, antibiotic, or antimetabolite.

48. The method of claim 41, wherein the chemotherapy comprises CPT-11, temozolomide, or a platin compound.

49. The method of claim 41, wherein radiotherapy comprises X-ray irradiation, UV-irradiation, γ-irradiation, or microwaves.

50. The method of claim 28, wherein from about 10³ to about 10¹⁵ viral particles are administered to the patient.

51-52. (canceled)

53. The method of claim 28, wherein the hyperproliferative disorder is a precancerous condition.

54. The method of claim 53, wherein the precancerous condition is cellular hyperplasia, metaplasia, or dysplasia.

55. The method of claim 28, wherein the hyperproliferative disorder is cancer.

56. The method of claim 55, wherein the cancer is a sarcoma, a metastatic cancer, a lymphatic metastases, a blood cell malignancy, a multiple myeloma, an acute leukemia, a chronic leukemia, a lymphoma, a head and neck cancer, a mouth cancer, a larynx cancer, a thyroid cancer, a lung cancer, a small cell carcinoma, a non-small cell cancer, a breast cancer, ductal carcinoma, gastrointestinal cancer, esophageal cancer, stomach cancer, colon cancer, colorectal cancer, pancreatic cancer, liver cancer, urologic cancer, bladder cancer, prostate cancer, ovarian carcinoma, uterine cancer, endometrial cancer, kidney cancer, renal cell carcinoma, brain cancer, neuroblastoma, astrocytic brain tumors, gliomas, metastatic tumor cell invasion in the central nervous system, bone cancers, osteomas, skin cancer, malignant melanoma, squamous cell carcinoma, basal cell carcinoma, hemangiopericytoma or Kaposi's sarcoma.

57. The method of claim 55, wherein the cancer is a recurrent cancer.

58. The method of claim 55, wherein the cancer is a refractory cancer.

59. The method of claim 55, wherein the cancer is a metastasis.

60-66. (canceled)
